(19) United States
(12) Patent Application Publication
(10) Pub. No.: US 2022/0193035 A1
(43) Pub. Date: Jun. 23, 2022

(54) INDOLE COMPOUNDS FOR USE IN NEURORESTORATION

(71) Applicant: Galimedix Therapeutics, Inc., Kensington, MD (US)

(72) Inventors: Christopher Graham Raphael. Parsons (Deceased), Nidderau (DE); Gerhard Rammes, Wielenbach (DE); Hermann Russ, Altendorf (CH); Andrew L Pearlman, Shorashim (IL)

(73) Assignee: Galimedix Therapeutics, Inc., Kensington, MD (US)

(21) Appl. No.: 17/605,489

(22) PCT Filed: Apr. 23, 2020
(86) PCT No.: PCT/US2020/029455

§ 371 Date: Oct. 21, 2021

Related U.S. Application Data
(60) Provisional application No. 62/837,741, filed on Apr. 24, 2019.

(51) Int. Cl. A61K 31/4045; A61P 25/28

(52) U.S. Cl. CPC A61K 31/4045; A61P 25/28

(57) ABSTRACT

Provided herein are methods of use of indole derivative compounds for reversal of amyloid β toxicity in amyloid β-associated diseases.

### FIELD OF INTEREST

Disclosed herein are methods to reverse Amyloid β (Aβ) toxicity, wherein the reversal of Aβ toxicity rapidly improves cell function in Aβ-associated diseases including Alzheimer's disease (AD), glaucoma, and macular degeneration of the retina.

### BACKGROUND

Amyloid β (Aβ)-associated diseases and conditions include diseases and conditions wherein neuronal and non-neuronal cell function is affected by the presence of toxic Aβ aggregates, which are formed from misfolded Aβ monomers by aggregation. Aβ-associated diseases and conditions include ophthalmic and neurological diseases and conditions for example but not limited to Alzheimer's disease (AD), glaucoma, and age-related macular degeneration of the retina. FIG. 1 provides a schematic showing the progression from normally folded Aβ-monomers to toxic Aβ oligomers.

AD is the most common form of dementia and its incidence is increasing at an alarming rate all over the world. The pathophysiology of AD is characterized by chronic, progressive neurodegeneration which involves early synaptotoxicity. One of the most obvious pathological features of AD is the accumulation of deposited Aβ in the brain. While normal Aβ is vital to proper neural function, misfolded versions of Aβ often associate with overproduction of Aβ, and are thought to underlie early synaptic pathology. Thus, reduction of toxic Aβ oligomers in the brain while not harming normal Aβ function, may be a promising therapeutic strategy in improving or reversing AD-related dysfunction.

Studies have shown that glaucoma is the second leading cause of blindness in the United States and is a neurodegenerative disease, with increasing evidence that Aβ toxicity plays an important role in its pathogenesis. The pathologic correlate of glaucoma is the progressive degeneration of retinal ganglion cells (RGC) and their axons which form the optic nerve. The classification of glaucoma includes the following different types: primary angle-closure glaucoma, secondary open-angle glaucoma, steroid-induced glaucoma, traumatic glaucoma, pigmentary dispersion syndrome, pseudoexfoliation syndrome, secondary angle-closure glaucoma, neovascular glaucoma, uveitis and glaucoma and other non further specified eye pathologies. Recently, Aβ has been found to co-localize with dying retinal ganglion cells. Animal studies also demonstrate that the soluble Aβ₁₋₄₂ oligomers, in particular, are very potent toxins for retinal ganglion cells. Thus, as with AD, Aβ toxicity is thought to play a pivotal role in glaucoma and its associated conditions.

Similarly, dry age-related macular degeneration of the retina (dry AMD) is a condition involving a pathology of the retina which has also been closely associated with the occurrence of Aβ toxicity in retinal pigment epithelium and photoreceptors, and which leads to a progressive loss of vision, leading finally to blindness.

According to the current understanding of the pathology in neurodegenerative diseases such as AD, glaucoma, and dry AMD, the affected neuronal or neurosensory cells suffer from the toxicity of the Aβ oligomers over time. These cells don't die immediately but rather, they enter first into a survival mode, with reduced metabolism and reduced membrane potential. In this state, for example in the retina, the cells don't function properly and thus they contribute less to the visual process, wherein the cells can reach a fully nonfunctional yet living state, which some authors refer to as “comatose cells”. A drug that can remove or reverse the toxic influence of the Aβ oligomers in the retina could potentially restore function in under-performing cells and transform comatose cells into fully functioning cells, thus increasing the number of cells and their net contribution to the visual process. The result of this reversal would be to improve the visual function of the patients. The same is true for comatose cells in the brain of Alzheimer patients, which suffer from the toxicity of Aβ oligomers and could similarly be restored to full function, leading to improved cognition. Such drugs are currently not available.

There exists a significant unmet medical need for methods which can reverse the symptoms caused by Aβ toxicity and restore the function of neurons and neurosensory cells in Aβ-associated neurodegenerative diseases, for example but not limited to dry AMD, glaucoma, and AD.

### SUMMARY

Disclosed herein are methods for use of a compound of Formula I for reversing amyloid β toxicity and rapidly restoring the function of neuronal, non-neuronal, or neuro-sensory cells, or a combination thereof in a subject in need, wherein a compound of Formula I or a non-toxic, non-β-sheet, amorphous amyloid β cluster, comprising amyloid β1-42 and the compound of Formula I, are administered to the subject in need.

In one aspect, disclosed herein are methods to reverse amyloid β toxicity and rapidly improve function of neuronal, non-neuronal, or neuro-sensory cells, or a combination thereof, in a subject in need, comprising administering to the subject a pharmaceutically effective amount of compound of Formula I

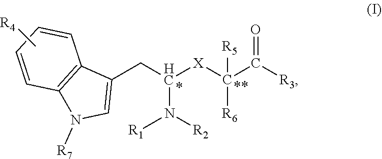

wherein
* refers to a chiral center;
* * refers to a chiral center if R₅ and R₆ are different;
R₁ is hydrogen, —C₁₋₆-alkyl, cycloC₃₋₁₂-alkyl, —C(O)R or —C(O)OR;
R₂ is hydrogen, C₁₋₆-alkyl, or cycloC₃₋₁₂-alkyl;
R₃ is —OR, —NHR or —N(R)₂;
R₄ is hydrogen, halogen, cyano, trifluoromethyl, —C₁₋₆-alkyl, —C₆₋₁₀-aryl, heteroaryl, —OR, —NHR, —N(R)₂, —C(O)R or —C(O)—NHR;
R⁵ is hydrogen, —C₁₋₆-alkyl or C₂₋₆-alkenyl; or
R⁵ and R⁶ together with the carbon atom carrying them form a cyclic system with 3 to 6 carbon atoms;
R⁶ is hydrogen, —C₁₋₆-alkyl or C₂₋₆-alkenyl;
R⁷ is hydrogen, methyl, ethyl, propyl or cyclopropyl;
R is hydrogen, —C₁₋₆-alkyl, or —C₆₋₁₀-aryl; and
X is a group —C(O)CH₂—, —CH(OH)CH₂—, —CH═CH—, —CH₂—NR—C(O)—, or —C(O)NR;
or an optical isomer, a pharmaceutically acceptable salt, a hydrate, a solvate, or a polymorph thereof.

In a related aspect, the compound of Formula I comprises Formula IA:

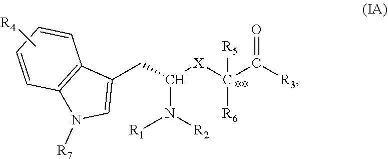

wherein variables R₁, R₂, R₃, R₄, R₅, R₆, R₇, and X are as for Formula I, or an optical isomer, a pharmaceutically acceptable salt, a hydrate, a solvate, or a polymorph thereof.

In another related aspect, the compound of Formula I or of Formula IA is selected from

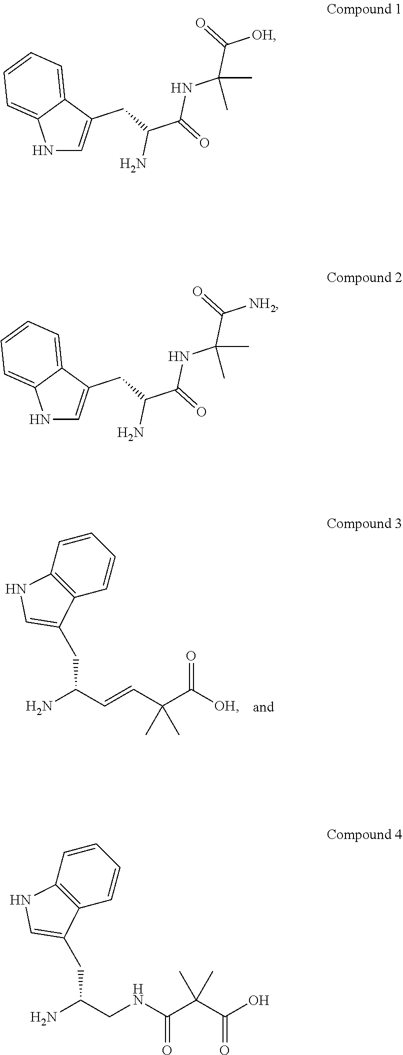

or a pharmaceutically acceptable salt, a hydrate, a solvate, or a polymorph thereof.

In another related aspect, the rapidly improved function of neuronal, non-neuronal, or neuro-sensory cells, or a combination thereof, comprises rapid restoration of impaired neuronal function, or decreased cell death of said neuronal, non-neuronal, or neuro-sensory cells, or a combination thereof. In a further related aspect, the neuronal, non-neuronal, or neuro-sensory cells comprise retinal ganglion cells (RGC), retinal pigment epithelium (RPE) cells, photosensory cells comprising rod and cone cells, hippocampal cells, or cortical cells, or a combination thereof.

In a related aspect, use of the methods disclosed herein comprises administering the compound of formula 1, or an optical isomer, a pharmaceutically acceptable salt, a hydrate, a solvate, or a polymorph thereof, to a subject that is suffering from an amyloid β associated disease. In another related aspect, the amyloid-beta associated disease comprises an ophthalmic or a neurological disease or condition. In further related aspect, the ophthalmic disease or condition comprises primary angle-closure glaucoma, secondary open-angle glaucoma, wide-angle glaucoma, steroid-induced glaucoma, traumatic glaucoma, pigmentary dispersion syndrome, pseudo-exfoliation syndrome, secondary angle-closure glaucoma, neovascular glaucoma, early and intermediate dry (non-exudative) age-related macular degeneration, macular degeneration with geographic atrophy, exudative (“wet”) macular degeneration, or diabetic retinopathy, or a combination thereof. In another further related aspect, the rapidly improved cell function comprises one or more aspects of visual function comprising visual acuity, low luminescence vision, contrast sensitivity, cone contrast sensitivity, color vision, focal and general retinal light sensitivity in photopic mesopic (light adaptation) and scotopic (dark adaptation) conditions, and postural stability balance and mobility, in said subject.

In a related aspect, the neurological disease or condition comprises type II diabetes mellitus, diabetes mellitus, Alzheimer's disease (AD), early onset Alzheimer's disease, late onset Alzheimer's disease, presymptomatic Alzheimer's disease, SAA amyloidosis, hereditary Icelandic syndrome, multiple myeloma, medullary carcinoma, aortic medical amyloid, Insulin injection amyloidosis, prion-systemic amyloidosis, chronic inflammation amyloidosis, senile systemic amyloidosis, pituitary gland amyloidosis, hereditary renal amyloidosis, familial British dementia, Finnish hereditary amyloidosis, familial non-neuropathic amyloidosis, and disorders and prion diseases, or a combination thereof. In a further related aspect, when said neurological disease comprises Alzheimer's disease (AD), early onset Alzheimer's disease, late onset Alzheimer's disease, or pre-symptomatic Alzheimer's disease, said rapid restoration of function comprises improvement of cognitive deficiencies, improvement of memory loss, reduction of abnormal behavior, reduction of hallucinations, reduction of loss of spatial orientation, reduction of apraxia, reduction of aggression, improvement in the ability to perform activities of daily living, or other symptoms of dementia, or any combination thereof, in said subject.

In a related aspect, administration comprises oral, topical, nasal, intravenous, subcutaneous, implanted slow-release depots, direct injection using an in-dwelling catheter, intrathecal injection, or intraocular injection administration. In a further related aspect, the administration is in the form of multiple doses administered over a period of time, wherein said time period comprises days, weeks, months, or years, or the lifetime of said subject. In a further related aspect, each dose comprises 100% or greater of the therapeutically effective dose. In another further related aspect, each dose comprises 20-75% of the therapeutically effective dose. In another further related aspect, individual doses of said multiple doses each comprise 100% of the therapeutically effective dose, 75-100% of the therapeutically effective dose, or 20-75% of the therapeutically effective dose, or any combination thereof. In another further related aspect, the pattern of dosage within the time period may be at regular intervals, irregular intervals, or a combination thereof comprising administration at regular and irregular intervals.

In a related aspect, the compound of Formula I comprises a non-toxic, non-β-sheet, amorphous Aβ cluster comprising amyloid β₁₋₄₂ and the compound of formula I.

In a related aspect, the compound of Formula I is comprised in a pharmaceutically acceptable composition.

Described herein in one aspect, is a method to reverse amyloid β toxicity and rapidly restore the function of neuronal, non-neuronal, or neuro-sensory cells, or a combination thereof, in a subject in need, said method comprising administration of a pharmaceutically effective amount of a non-toxic, non-β-sheet, amorphous amyloid β cluster, said cluster comprising amyloid β₁₋₄₂:compound of Formula I at a ratio of about 500:1, wherein the compound of Formula I is represented by the following structure

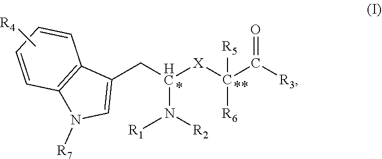

wherein
* refers to a chiral center;
* * refers to a chiral center if R₅ and R₆ are different;
R₁ is hydrogen, —C₁₋₆-alkyl, cycloC₃₋₁₂-alkyl, —C(O)R or —C(O)OR;
R₂ is hydrogen, C₁₋₆-alkyl, or cycloC₃₋₁₂-alkyl;
R₃ is —OR, —NHR or —N(R)₂;
R₄ is hydrogen, halogen, cyano, trifluoromethyl, —C₁₋₆-alkyl, —C₆₋₁₀-aryl, heteroaryl, —OR, —NHR, —N(R)₂, —C(O)R or —C(O)—NHR;
R⁵ is hydrogen, —C₁₋₆-alkyl or C₂₋₆-alkenyl; or
R⁵ and R⁶ together with the carbon atom carrying them form a cyclic system with 3 to 6 carbon atoms;
R⁶ is hydrogen, —C₁₋₆-alkyl or C₂₋₆-alkenyl;
R⁷ is hydrogen, methyl, ethyl, propyl or cyclopropyl;
R is hydrogen, —C₁₋₆-alkyl, or —C₆₋₁₀-aryl; and
X is a group —C(O)CH₂—, —CH(OH)CH₂—, —CH═CH—, —CH₂—NR—C(O)—, or —C(O)NR;
or an optical isomer, a pharmaceutically acceptable salt, a hydrate, a solvate, or a polymorph thereof.

In a related aspect, the concentration of amyloid β₁₋₄₂ is about 50 nM and the concentration of the compound of Formula I is about 0.1 nM. In a further related aspect, the compound is comprised in a pharmaceutically acceptable composition.

In a related aspect, the non-toxic, non-β-sheet, amorphous amyloid β cluster is produced by a method comprising serially diluting the compound of Formula 1 in solutions of amyloid β 1-42 said method comprising stepwise dilution of the compound of Formula 1 to a final concentration of 0.1 nM. In a further related aspect, the stepwise dilution comprises 5 serial dilution steps.

### BRIEF DESCRIPTION OF THE DRAWINGS

The subject matter disclosed herein related to methods of use of compounds of Formula I is particularly pointed out and distinctly claimed in the concluding portion of the specification. The methods of use, however, both as to organization and method of operation, together with objects, features, and advantages thereof, may best be understood by reference to the following detailed description when read with the accompanying drawings in which:

FIG. 1 shows a schematic of the progression of amyloid β (Aβ) monomers to toxic Aβ oligomers, wherein a compound of Formula I or a non-toxic, non-β-sheet, amorphous Aβ cluster comprising Aβ₁₋₄₂ and the compound of Formula I, triggers the aggregation of misfolded Aβ to form non-toxic, non-β-sheet, amorphous Aβ clusters, and reverses the formation of toxic Aβ oligomers, wherein the detoxification of the misfolded Aβ monomers and toxic Aβ oligomers occurs in the absence or near absence of the compound of Formula I through a self-propagating process. In some embodiments, the compound of Formula I comprises Compound 1.

FIGS. 2A-2B show the neurorestorative effect of Compound 1 in hippocampal tissue, illustrated by the results of extracellular—dual input Long Term Potentiation (LTP) recordings in hippocampal slices with two stimulating electrodes. FIG. 2A shows LTP recordings under two sequential conditions from the same hippocampal tissue slice—namely, the first condition was Amyloid β₁₋₄₂ (Aβ₁₋₄₂) alone (50 nM; black circles) and the second condition was Amyloid β₁₋₄₂ (50 nM) together with Compound 1 (0.1 nM after serial dilution (SD); grey circles). Aβ₁₋₄₂ 50 nM was applied via the bath solution for 90 min (only last 20 mins of this baseline are shown) before attempting to induce LTP following high frequency tetanus at 100 Hz for 1 sec delivered via the first electrode. After recording LTP for 60 mins, the bath solution was exchanged for that following serial dilution with Compound 1. This solution still contained Aβ₁₋₄₂ 50 nM but together with a final concentration of 0.1 nM of Compound 1. This was incubated for a further 90 mins (again only last 20 mins of this second baseline are shown) before attempting to induced LTP in the second input which was then recorded for an additional 60 mins. FIG. 2B shows the percent potentiation for the final 10 minutes under each field Excitatory Post Synaptic Potential (fEPSP) recording condition—namely 50 nM Amyloid β₁₋₄₂, alone—black bar; 50 nM Amyloid β₁₋₄₂ and 0.1 nM Compound 1, prepared by serial dilution (DS=dilution series—grey bar).

FIGS. 3A-3B show the neurorestorative effect of Compound 2 in hippocampal tissue, illustrated by the results of extracellular—dual input Long Term Potentiation (LTP) recordings in hippocampal slices with two stimulating electrodes. FIG. 3A shows LTP recordings under two sequential conditions from the same hippocampal tissue slice—namely, the first condition was Amyloid β₁₋₄₂ (Aβ₁₋₄₂) alone (50 nM; black circles) and the second condition was Amyloid β₁₋₄₂ (50 nM) together with Compound 2 (0.1 nM after serial dilution (SD); grey circles). Aβ₁₋₄₂ 50 nM was applied via the bath solution for 90 min (only last 20 mins of this baseline are shown) before attempting to induce LTP following high frequency tetanus at 100 Hz for 1 sec delivered via the first electrode. After recording LTP for 60 mins, the bath solution was exchanged for that following serial dilution with Compound 2. This solution still contained Aβ₁₋₄₂ 50 nM but together with a final concentration of 0.1 nM of Compound 2. This was incubated for a further 90 mins (again only last 20 mins of this second baseline are shown) before attempting to induced LTP in the second input which was then recorded for an additional 60 mins. FIG. 3B shows the percent potentiation for the final 10 minutes under each field Excitatory Post Synaptic Potential (fEPSP) recording condition—namely 50 nM Amyloid β₁₋₄₂, alone—black bar; 50 nM Amyloid β₁₋₄₂ and 0.1 nM Compound 2, prepared by serial dilution (DS=dilution series—grey bar).

FIGS. 4A-4B show elevated Amyloid β₁₋₄₂ (Aβ) in the retina of Glaucoma Patients. FIG. 4A presents data comparing Amyloid β₁₋₄₂ in control (n=5) and glaucoma (n=5) patients. FIG. 4B presents immunostaining of retinal sections showing the localization of Aβ (red fluorescence) in glaucoma patients' retinal ganglion cells (arrow), which represent the retina layer affected in glaucoma. Aβ is also seen in the optic nerve fiber layer (triangles) of the glaucoma patients.

FIGS. 5A-5B show data demonstrating Compound 1 provides dose-dependent reduction in toxic Amyloid β₁₋₄₂ in the retina (photoreceptor layer) of a mouse model, which simulates age-related macular degeneration (AMD; early intermediate AMD). FIG. 5A presents a bar-graph showing the results of 3 months' daily treatment of 5-6 month-old AMD mice (genetic model which accumulate Amyloid β₁₋₄₂ in the photoreceptor layer of the retina. Eye-drops comprising one of two doses of Compound 1, were administered three times every day. Control eye-drops comprised the vehicle alone. Significant reduction of deposited Amyloid 3 is observed using eye-drops containing 0.5% or 2.0% of Compound 1, versus control. FIG. 5B presents immunostaining in a series of retinal sections of 24-month old C57BL/6 mice with Aβ₁₋₄₂ and C3b aggregation. (Red is Aβ; yellow/green is C3b). From month 23, the mice were treated trice daily with either control (vehicle only), 0.5% or 2% Compound 1. In the control mouse, deposited Aβ was thick and linear along the Bruch's membrane with diffuse staining in the retinal pigment epithelium (RPE) above it, but was very significantly reduced in the mice treated with either of two different concentrations of Compound 1 (HD1 0.5% and HD2 2.0%), showing only isolated aggregates (circled) and no staining in the RPE. Corresponding patterns in the yellow/green staining of the same section indicated colocalized reduction in C3b response in the eyes treated with Compound 1, versus control. As C3b is thought to be in response to inflammation caused by toxic Aβ, the reduced C3b staining is likely due to the reduction in toxic Aβ caused by Compound 1. Scale bar=10 μm.

FIG. 6 presents a schematic of one embodiment of the serial dilution of Compound 1 (Cmpd 1) with Aβ, wherein Compound 1 is serially diluted from 1 μM to 0.1 nM while the concentration of Aβ is maintained at 50 nM. After incubating the Aβ₁₋₄₂:Compound 1 mixture for 20 mins., 10% (5 mL) was transferred to a freshly prepared solution with Aβ (50 nM). This dilution step was repeated 5 times finally resulting in a 1000:1 stoichiometric excess of Aβ₁₋₄₂ over Compound 1.

### DETAILED DESCRIPTION

In the following detailed description, numerous specific details are set forth in order to provide a thorough understanding of indole derivative the compounds of Formula I and uses thereof for neurorestoration in subjects suffering from an amyloid-β (Aβ) associated disease. In certain instances, well-known methods, procedures, and components have not been described in detail so as not to obscure the present disclosure.

Methods of use disclosed herein, reverse Aβ functional toxicity of neuronal, non-neuronal, and neuro-sensory cells in a subject in need. Methods reversing Aβ functional toxicity may in some embodiments, provide symptomatic treatment, thereby improving a function or functions in the subject in need. In some embodiments, the improved function comprises a function damaged, reduced, inhibited, or altered in an amyloid β-associated disease or condition.

Methods of use disclosed herein, reverse Aβ toxicity of neuronal, non-neuronal, and neuro-sensory cells in a subject in need. Methods reversing Aβ toxicity may in some embodiments, provide symptomatic treatment, thereby improving a function or functions in the subject in need. In some embodiments, the improved function comprises a function damaged, reduced, inhibited, or altered in an amyloid β-associated disease or condition. In some embodiments, methods disclosed herein reverse Aβ toxicity and rapidly improve function of neuronal, non-neuronal, or neurosensory cells, or a combination thereof.

Methods reversing amyloid β functional toxicity, in some embodiments comprise a step administering indole derivatives, or optical isomers, pharmaceutically acceptable salts, hydrates, solvates, or polymorphs thereof, or compositions thereof. Methods reversing amyloid β toxicity, in some embodiments comprise a step administering indole derivatives, or optical isomers, pharmaceutically acceptable salts, hydrates, solvates, or polymorphs thereof, or compositions thereof Disclosed herein, are some embodiments of indole derivatives, or optical isomers, pharmaceutically acceptable salts, hydrates, solvates, or polymorphs thereof, or compositions thereof, that reverse amyloid β functional toxicity of neuronal, non-neuronal, and neuro-sensory cells. In some embodiments, indole derivatives disclosed herein, or optical isomers, pharmaceutically acceptable salts, hydrates, solvates, or polymorphs thereof, or compositions thereof provide symptomatic treatment for an amyloid β-associated disease or condition. In some embodiments, indole derivatives disclosed herein, or optical isomers, pharmaceutically acceptable salts, hydrates, solvates, or polymorphs thereof, or compositions thereof improve functionality of a symptom in a subject suffering from an amyloid β-associated disease or condition.

In some embodiments, the terms “amyloid β”, “Aβ peptide”, “Aβ₁₋₄₂”, and “Aβ” are interchangeable, having the same meaning and qualities. Aβ₁₋₄₂ is one example of a toxic Aβ peptide. The more common, but somewhat less toxic form of an Aβ peptide is, for example, Aβ₁₋₄₀. There are also other length peptides, as well as post-translationally modified forms, some of which are even claimed to be more toxic than Aβ₁₋₄₂. While Aβ₁₋₄₂ is considered the most toxic form of Aβ, other forms exist. The skilled artisan would appreciate that reference to “Aβ” encompasses the toxic form of an amyloid β peptide. In some embodiments, Aβ comprises Aβ₁₋₄₂ peptide. In some embodiments, Aβ comprises Aβ₁₋₄₂ peptide plus other forms of toxic Aβ peptides. In contrast, the term “Aβ clusters”, encompasses non-toxic, non-β-sheet, amorphous Aβ cluster formations.

Disclosed herein are methods of reversing amyloid β functional toxicity of neuronal, non-neuronal, and neuro-sensory cells in a subject in need, comprising administration of a pharmaceutically effective amount of Compound of Formula I

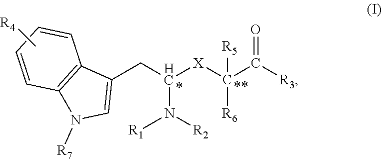

wherein* refers to a chiral center;

** refers to a chiral center if R₅ and R₆ are different;

R₁ is hydrogen, —C₁₋₆-alkyl, cycloC₃₋₁₂-alkyl, —C(O)R, or —C(O)OR;

R₂ is hydrogen, C₁₋₆-alkyl, or cycloC₃₋₁₂-alkyl;

R₃ is —OR, —NHR, or —N(R)₂;

R₄ is hydrogen, halogen, cyano, trifluoromethyl, —C₁₋₆-alkyl, —C₆₋₁₀-aryl, heteroaryl, —OR, —NHR, —N(R)₂, —C(O)R, or —C(O)—NHR;

R⁵ is hydrogen, —C₁₋₆-alkyl, or C₂₋₆-alkenyl; or

R⁵ and R⁶ together with the carbon atom carrying them form a cyclic system with 3 to 6 carbon atoms;

R⁶ is hydrogen, —C₁₋₆-alkyl, or C₂₋₆-alkenyl;

R is hydrogen, —C₁₋₆-alkyl, or —C₆₋₁₀-aryl;

X is —C(O)CH₂—, —CH(OH)CH₂—, —CH═CH—, —CH₂—NR—C(O)—, or —C(O)NR—;

R⁷ is hydrogen, methyl, ethyl, propyl, or cyclopropyl,

or an optical isomer, a pharmaceutically acceptable salt, a hydrate, a solvate, or a polymorph thereof.

As indicated by the * and ** in Formula I, compounds comprising a structure of Formula I may comprise at least one and possibly 2 chiral centers. Each of * and ** independently denotes either (R) configuration or (S) configuration. One of the main obstacles in using short peptide-like fragments in therapy is their proteolytic degradation by stereospecific cellular proteases. There may therefore be an advantage to using one stereoisomer over another in methods of treatment disclosed herein in order to avoid metabolism of the active component of the treatment by specific stereospecific proteases. In some embodiments of methods disclosed herein, one or both optional asymmetric carbons (marked by * and ** in Formula I) have an (R) configuration. In some embodiments of methods disclosed herein, the asymmetric carbon (marked by * in Formula I) has an (R) configuration.

Disclosed herein are methods of reversing amyloid β toxicity and rapidly improving the function of neuronal, non-neuronal, or neuro-sensory cells, or a combination thereof, in a subject in need, comprising administration of a pharmaceutically effective amount of Compound of Formula I

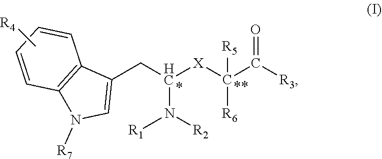

wherein* refers to a chiral center;

** refers to a chiral center if R₅ and R₆ are different;

R₁ is hydrogen, —C₁₋₆-alkyl, cycloC₃₋₁₂-alkyl, —C(O)R, or —C(O)OR;

R₂ is hydrogen, C₁₋₆-alkyl, or cycloC₃₋₁₂-alkyl;

R₃ is —OR, —NHR, or —N(R)₂;

R₄ is hydrogen, halogen, cyano, trifluoromethyl, —C₁₋₆-alkyl, —C₆₋₁₀-aryl, heteroaryl, —OR, —NHR, —N(R)₂, —C(O)R, or —C(O)—NHR;

R⁵ is hydrogen, —C₁₋₆-alkyl, or C₂₋₆-alkenyl; or

R⁵ and R⁶ together with the carbon atom carrying them form a cyclic system with 3 to 6 carbon atoms;

R⁶ is hydrogen, —C₁₋₆-alkyl, or C₂₋₆-alkenyl;

R is hydrogen, —C₁₋₆-alkyl, or —C₆₋₁₀-aryl;

X is —C(O)CH₂—, —CH(OH)CH₂—, —CH═CH—, —CH₂—NR—C(O)—, or —C(O)NR—;

R⁷ is hydrogen, methyl, ethyl, propyl, or cyclopropyl,

or an optical isomer, a pharmaceutically acceptable salt, a hydrate, a solvate, or a polymorph thereof.

In some embodiments the * carbon is an asymmetric carbon that has an (R) configuration. In some embodiments, a method disclosed herein comprises use of Compound of Formula IA:

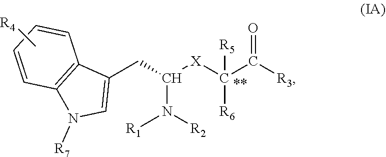

wherein variables R1, R2, R3, R4, R5, R6, R7, and X are defined for the structure of formula I, or an optical isomer, a pharmaceutically acceptable salt, a hydrate, a solvate, or a polymorph thereof.

Methods of reversing amyloid β toxicity or functional toxicity of neuronal and neuro-sensory cells using the compounds disclosed herein, may be beneficial for reversing the course of an amyloid-associated disease or disorder in a subject in need thereof. A skilled artisan would appreciate that reversing the course of an amyloid-associated disease or disorder may encompass (1) a reduction of amyloid plaque depositions present in a pathological state; (2) a reversal of neuronal and or neurosensory cell functionality, for example but not limited to a reversal of long term potentiation in neuronal and or neurosensory cells; (3) a neurorestoration of neuronal and or neurosensory cell functionality, for example but not limited to enhancing the long term potentiation in neuronal and or neurosensory cells present in a pathological condition; (4) a neurorestoration of neuronal and or neurosensory cell functionality, for example but not limited to improving visual acuity, low luminescence vision, or retinal light sensitivity, or a combination thereof in a subject suffering from an ophthalmic amyloid β pathological condition, such as but not limited to glaucoma or dry-eye age-related macular degeneration; or (5) a neurorestoration of neuronal and or neurosensory cell functionality, for example but not limited to improving cognitive deficiencies, memory loss, and ability to perform activities of daily living, or a combination thereof, in a subject suffering from a neurological amyloid β pathological condition, such as but not limited to Alzheimer's Disease; or combinations thereof. In some embodiments, along with a reduction of amyloid plaques, reversing the course of an amyloid-associated disease or disorder may encompass a reduction of drusen. Drusen may form under the retina and or in the optic nerve in dry AMD.

Prior to describing the methods of use of the compounds disclosed here, the section below provides a description of the indole derivative compounds disclosed herein.

### Indole Derivatives

In one embodiment, the methods disclosed herein make use of a compound represented by the structure of formula I.

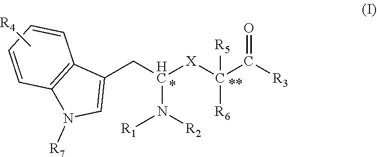

wherein:

* refers to a chiral center;

** refers to a chiral center if R₅ and R₆ are different;

R₁ is hydrogen, —C₁₋₆-alkyl, cycloC₃₋₁₂-alkyl, —C(O)R, or —C(O)OR;

R₂ is hydrogen, C₁₋₆-alkyl, or cycloC₃₋₁₂-alkyl;

R₃ is —OR, —NHR, or —N(R)₂;

R₄ is hydrogen, halogen, cyano, trifluoromethyl, —C₁₋₆-alkyl, —C₆₋₁₀-aryl, heteroaryl, —OR, —NHR, —N(R)₂, —C(O)R, or —C(O)—NHR;

R⁵ is hydrogen, —C₁₋₆-alkyl, or C₂₋₆-alkenyl; or

R⁵ and R⁶ together with the carbon atom carrying them form a cyclic system with 3 to 6 carbon atoms;

R⁶ is hydrogen, —C₁₋₆-alkyl, or C₂₋₆-alkenyl;

R is hydrogen, —C₁₋₆-alkyl, or —C₆₋₁₀-aryl;

X is —C(O)CH₂—, —CH(OH)CH₂—, —CH═CH—, —CH₂—NR—C(O)—, or —C(O)NR—;

R⁷ is hydrogen, methyl, ethyl, propyl, or cyclopropyl,

or an optical isomer, pharmaceutically acceptable salt, hydrate, solvate, or polymorph thereof.

In one embodiment, the methods disclosed herein make use of a compound represented by the structure of formula IA.

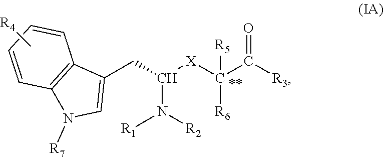

wherein variables R₁, R₂, R₃, R₄, R₅, R₆, R₇, and X are defined for the structure of formula I.

In one embodiment, the methods disclosed herein make use of a compound represented by the structure of formula II:

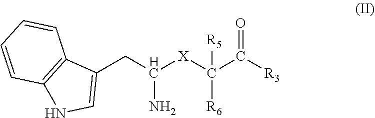

wherein variables R₃, R₅, R₆ and X are defined for the structure of formula I.

In one embodiment, the methods disclosed herein make use of a compound represented by the structure of formula IIA:

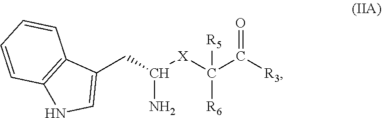

wherein variables R₃, R₅, R₆ and X are defined for the structure of formula I.

In one embodiment, R₁ is hydrogen. In another embodiment, R₁ is —C₁₋₆-alkyl. In one embodiment, R₁ is —C(O)R. In certain embodiment, R₁ is —C(O)—CH₃. In one embodiment, R₁ is —C(O)-t-butyl. In one embodiment, R₁ is —C(O)-2,2-dimethylpropyl. In one embodiment, R₁ is —C(O)OR. In another embodiment, R₁ is —C(O)OCH₃.

In one embodiment, R₂ is hydrogen. In another embodiment, R₂ is —C₁₋₆-alkyl.

In one embodiment, R₁ is hydrogen and R₂ is hydrogen. In another embodiment, R₁ is —C(O)R and R₂ is hydrogen.

In one embodiment, R₃ is —OH. In one embodiment, R₃ is —OCH₃. In one embodiment, R₃ is —NH₂. In one embodiment, R₃ is —NH—CH₃. In one embodiment, R₃ is —NH-t-butyl. In one embodiment, R₃ is —N(CH₃)₂.

In some embodiments, R₁ and R₂ is each independently hydrogen or C₁₋₃-alkyl.

In one embodiment, R₄ is hydrogen.

In one embodiment, R₅ is hydrogen or —C₁₋₆-alkyl. In one embodiment, R₆ is hydrogen or —C₁₋₆-alkyl.

In one embodiment, R₅ and R₆ are identical. In one embodiment, R₅ and R₆ are —CH₃.

In one embodiment, the two substituents R₅ and R₆ can, together with the carbon atom carrying them, form a cyclic system with 3 to 6 carbon atoms. In one embodiment, this cyclic system can contain one ring element selected from the group consisting of —O—, —S—, and —NH—. In one embodiment, the cyclic systems include, but are not limited to, cyclohexane, cyclopentane, cyclobutane, cyclopropane, oxetane, and acetidine rings.

In one embodiment, X is —C(O)CH₂—, —CH(OH)CH₂—, —CH═CH—, —CH₂CH₂NRC(O)—, or —C(O) NR—. In one embodiment, X represents —CH═CH—. In one embodiment, X represents CH₂NRC(O)—. In one embodiment, X represents —C(O)NR—.

In one embodiment, group X as indicated has an orientation of the left side being connected with the chiral carbon atom carrying the amino group.

In one embodiment, R⁷ is hydrogen or methyl. In one embodiment, R⁷ is hydrogen.

In one embodiment, the compound for use in the methods disclosed herein includes all optical isomers, pharmaceutically acceptable salts, hydrates, solvates and polymorphs of the compounds of Formula (I), (IA), (II) or (IIA). The compounds for use described herein also relates to analogs and derivatives of compounds of Formula (I), (IA), (II) or (IIA).

As used herein, in one embodiment, the term “C₁₋₆-alkyl” represents straight or branched chain alkyl groups such as methyl, ethyl, n-propyl, 2-propyl, n-butyl and tert-butyl. The alkyl group, in one embodiment, may be optionally substituted by one to five substituents selected from halogen, amino, hydroxyl, and —CF₃.

As used herein, in one embodiment, the term “C₂₋₆-alkenyl” represents straight or branched chain alkenyl groups.

As used herein, in one embodiment, the term “cycloC₃₋₁₂-alkyl” represents monocyclic or bicyclic alkyl groups, including cyclopropyl, cyclobutyl, cyclopentyl, and cyclohexyl. The cycloalkyl groups, in one embodiment, may be optionally substituted by one to five substituents selected from C₁₋₆-alkyl, halogen, amino, and hydroxyl.

As used herein, in one embodiment, the term “C₆₋₁₀-aryl” represents phenyl or naphthyl, wherein the phenyl or naphthyl group, in one embodiment, may be optionally substituted by one to five substituents selected from C₁₋₆-alkyl, cycloC₃₋₁₂-alkyl, halogen, amino, and hydroxyl.

As used herein, in one embodiment, the term “heteroaryl” represents an aromatic 5-6 membered ring containing from one to four heteroatoms selected from oxygen, sulfur, and nitrogen, or a bicyclic group comprising a 5-6 membered ring containing from one to four heteroatoms selected from oxygen, sulfur, and nitrogen fused with a benzene ring or a 5-6 membered ring containing from one to four heteroatoms selected from oxygen, sulfur and nitrogen, wherein the heteroaryl group, in one embodiment, may be optionally substituted by one or two substituents selected from C₁₋₆-alkyl, cycloC₃₋₁₂-alkyl, halogen, amino, hydroxyl.

As used herein, in one embodiment, the term “halogen” represents fluorine, chlorine, bromine and iodine.

In some embodiments, compounds described herein may be in the form of pharmaceutically acceptable salts. A skilled artisan would appreciate that “pharmaceutically acceptable salts” refers to those salts which possess the biological effectiveness and properties of the parent compound and which are not biologically or otherwise undesirable. The nature of the salt or isomer is not critical, provided that it is non-toxic and does not substantially interfere with the desired pharmacological activity

As used herein, in one embodiment, the term “analog” or “derivative” refers to a molecule that structurally resembles a reference molecule but has been modified in a targeted and controlled manner to replace one or more specific substituents of the referent molecule with an alternate substituent, thereby generating a molecule which is structurally similar to the reference molecule. Synthesis and screening of analogs (e.g., using structural or biochemical analysis) to identify slightly modified versions of a known compound which may have improved properties (e.g., higher potency and/or selectivity at a specific targeted receptor/protein type, greater ability to penetrate into the eye, fewer side effects) is a typical drug design approach.

In one embodiment, the compound of formula (I) or (IA) for use in the methods disclosed herein is represented by:

R₁ is hydrogen, —C₁₋₆-alkyl, —C(O)—R or —C(O)—OR;

R₂ is hydrogen or —C₁₋₆-alkyl;

R₃ is —OR, —NHR, or —NR₂;

R₄ is hydrogen, halogen, cyano, trifluoromethyl, —C₁₋₆-alkyl;

R₅ is hydrogen or —C₁₋₆-alkyl; in particular —C₁₋₃-alkyl;

R₆ is hydrogen or —C₁₋₆-alkyl; in particular —C₁₋₃-alkyl; or

R₅ and R₆ together with the carbon atom carrying them form a cyclic system with 3 to 6 carbon atoms;

R is hydrogen or —C₁₋₆-alkyl; in particular hydrogen or —C₁₋₃-alkyl;

X is —C(O)CH₂—, —CH═CH—, or —CH₂NRC(O)—, or —C(O)NR;

R₇ is hydrogen or methyl;

or an optical isomer, pharmaceutically acceptable salt, hydrate, solvate, or polymorph thereof.

In one embodiment, in the compound of formula (I) or (IA) for use in the methods disclosed herein is represented by,

R₁ is hydrogen, —C₁₋₃-alkyl, or —C(O)—CH₃;

R₂ is hydrogen or —C₁₋₃-alkyl;

R₃ is —OR, —NHR, or —NR₂;

R₄ is hydrogen or halogen;

R₅ is —C₁₋₃-alkyl;

R₆ is —C₁₋₃-alkyl;

R is hydrogen or —C₁₋₃-alkyl;

X is —C(O)CH₂—, —CH═CH—, or —CH₂NRC(O)—, or —C(O)NR—;

R₇ is hydrogen;
or an optical isomer, pharmaceutically acceptable salt, hydrate, solvate, or polymorph thereof.

In one embodiment, the compound of formula (I) or (IA) for use in the methods disclosed herein is represented by,

R₁ is hydrogen, —C₁₋₃-alkyl, or —C(O)—CH₃;

R₂ is hydrogen;

R₃ is —OR or —NHR;

R₄ is hydrogen;

R₅ is hydrogen or —C₁₋₃-alkyl;

R₆ is hydrogen or —C₁₋₃-alkyl;

R is hydrogen or —C₁₋₃-alkyl;

X is —C(O)CH₂—, —CH═CH—, or —CH₂NRC(O)—, or —C(O)NR—;

R₇ is hydrogen;

or an optical isomer, pharmaceutically acceptable salt, hydrate, solvate, or polymorph thereof.

In one embodiment, the compound of formula (I) or (IA) for use in the methods disclosed herein is represented by,

R₁ is hydrogen or —C(O)—CH₃;
R₂ is hydrogen;

### R₃ is —OR or —NHR;

R₄ is hydrogen;
R₅ is —C₁₋₃-alkyl;
R₆ is —C₁₋₃-alkyl;
R is hydrogen or —C₁₋₃-alkyl;
X is —C(O)CH₂—, —CH═CH—, or —CH₂NRC(O)—, or —C(O)NR—;
R₇ is hydrogen;
or an optical isomer, pharmaceutically acceptable salt, hydrate, solvate, or polymorph thereof.

In one embodiment, the term “optical isomer” is meant to encompass optical isomers of an indole derivative compound of Formulae I, IA, II, or IIA. It will be appreciated by those skilled in the art that the indole derivative compounds described herein, may contain at least one chiral center. Accordingly, the indole derivative compounds used in the methods disclosed herein may exist in, and be isolated in, optically-active or racemic forms. Some compounds may also exhibit polymorphism. It is to be understood that use of the compounds disclosed herein encompasses methods of use any racemic, optically-active, polymorphic, or stereoisomeric form, or mixtures thereof, which form possesses properties useful in the treatment of amyloid β diseases or conditions described herein.

In another embodiment, methods of use disclosed herein include uses of hydrates of the compounds of Formula I, IA, II, IIA, and any of compounds 1-25. In one embodiment, the term “hydrate” refers to hemihydrate, monohydrate, dihydrate, trihydrate or others, as known in the art.

In one embodiment, in the compound of formula (I) or formula (II) for the method disclosed herein, the chiral center carrying the amino group and the group X has R-configuration.

In one embodiment, the compound for use in the methods disclosed herein is represented by compounds 1-4:

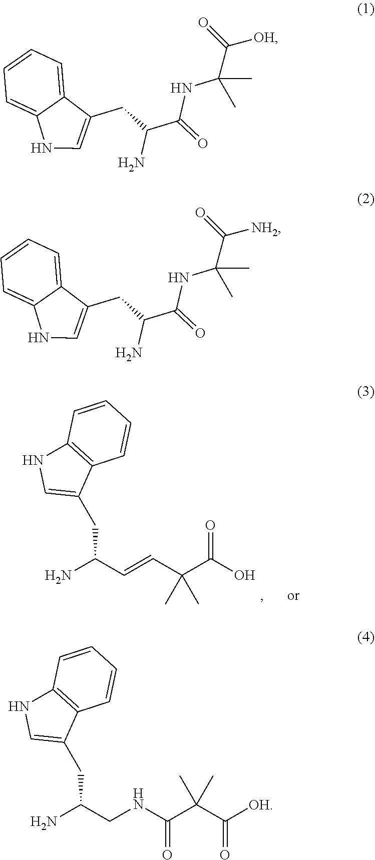

In one embodiment, the compound for use in the methods disclosed herein is represented by compounds, 1, 2, 3, or 4, or pharmaceutically acceptable salt, hydrate, solvate, or polymorph thereof.

In one embodiment, the compound for use in the method disclosed herein is represented by compound 5-25:

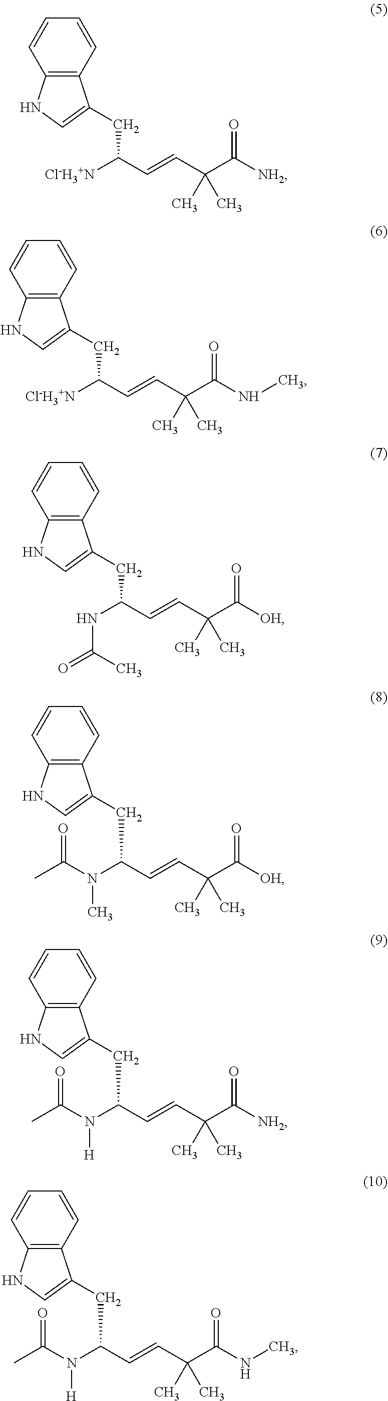

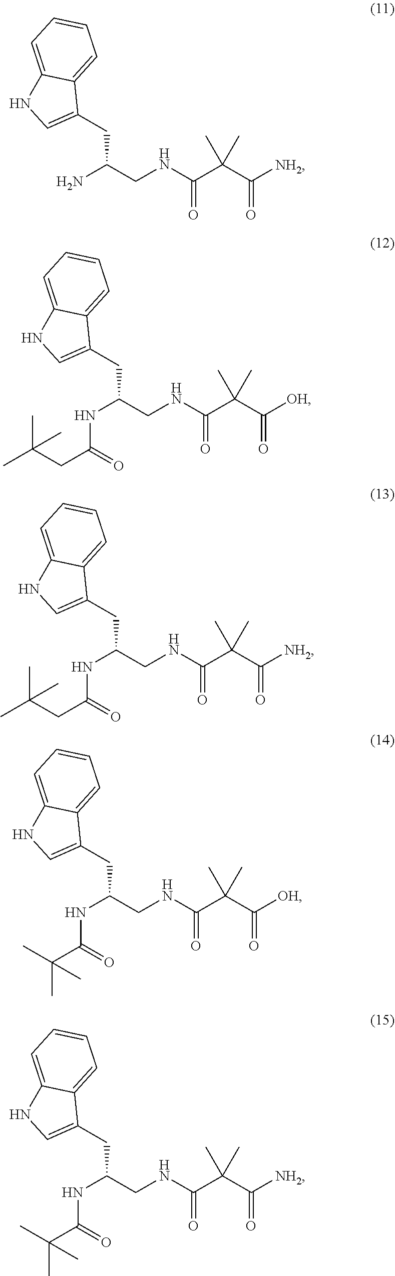

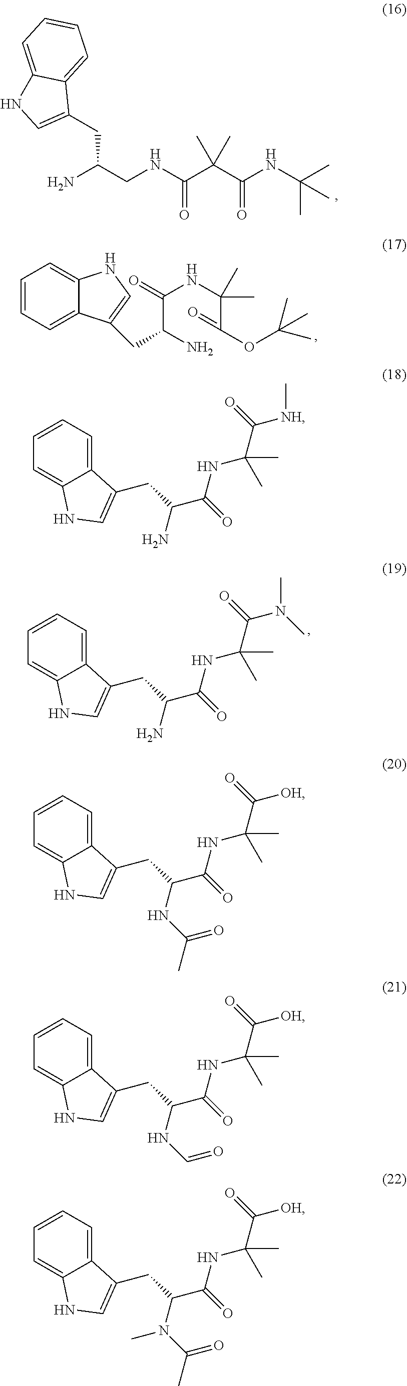

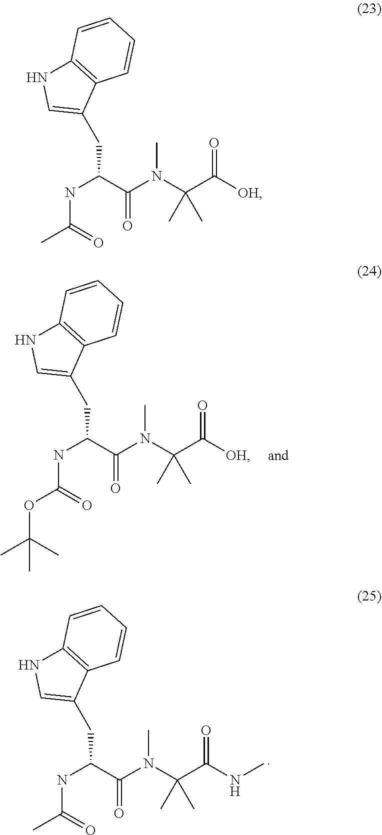

In one embodiment, the compound for use in the methods disclosed herein is represented by any of compounds 5-25 or pharmaceutically acceptable salt, hydrate, solvate, or polymorph thereof.

In one embodiment, the compound for use in the methods disclosed herein is represented by compounds, 5, 6, 7, 8, 9, 10, 11, 12, 13, 14, 15, 16, 17, 18, 19, 20, 21, 22, 23, 24, or 25, or pharmaceutically acceptable salt, hydrate, solvate, or polymorph thereof.

The compounds for use described herein also relates to analogs and derivatives of compounds 1, 2, 3, 4, 5, 6, 7, 8, 9, 10, 11, 12, 13, 14, 15, 16, 17, 18, 19, 20, 21, 22, 23, 24, or 25.

In some embodiments, a method disclosed herein for reversing Aβ toxicity and for rapidly improving the function of neuronal, non-neuronal, or neurosensory cells, or a combination thereof comprises administering a non-toxic, non-β-sheet, amorphous Aβ cluster comprising a compound of Formula 1, or an optical isomer, pharmaceutically acceptable salt, hydrate, solvate, or polymorph as described in detail herein above, and Aβ₁₋₄₂.

Preparation of Compounds

The compound for use in the methods disclosed herein, for example compounds of Formula I, IA, II, and IIA, or an optical isomer, pharmaceutically acceptable salt, hydrate, solvate, or polymorph thereof, can be prepared by the methods known in the art. In one embodiment, the compound for use in the methods described herein can be prepared based on the preparation procedures as described in the published applications such as WO2012066549, WO 2012/055945 A1, and WO 2012/066549 A1.

For example, in some embodiments, the peptide D-Trp-Aib, which herein is referred to as Compound 1, may be synthesized as presented in International Publication No. WO2012066549 at Example 1, and Frydman-Marom, A., Rechter, M., Shefler, I., Bram, Y., Shalev, D. E. and Gazit, E. (2009). Cognitive-performance recovery of Alzheimer's disease model mice by modulation of early soluble amyloidal clusters. Angew Chem Int Ed Engl 48(11): 1981-1986, supplementary information, which are both incorporated herein in full. Briefly, D-Trp-Aib synthesis was as follows: The peptide was synthesized according to classical liquid phase peptide synthesis, using customized protocols involved standard amide bond formation method, namely the protection of N-terminal amine and C-terminal carboxylic function, coupling of two protected amino acids and cleavage of the protecting groups to obtain the desired product in free peptide form. The crude product was purified by reverse phase preparative HPLC, the purity was determined by reverse phase analytical HPLC analysis (>95%) and the structure was confirmed by mass spectrometry (MW 289.33).

International Application publication WO 2012/066549 describes some embodiments of the synthesis of Compound 2. In WO 2012/066549, Compound 2 described herein, is termed compound “D”. (See WO 2012/066549 at Example 1 description of compounds prepared using Scheme 8.) The description presented in WO 2012/066549 of the synthesis of Compound 2 is incorporated herein in its entirety. Briefly, Compound 2 was prepared as depicted in Scheme 1 below.

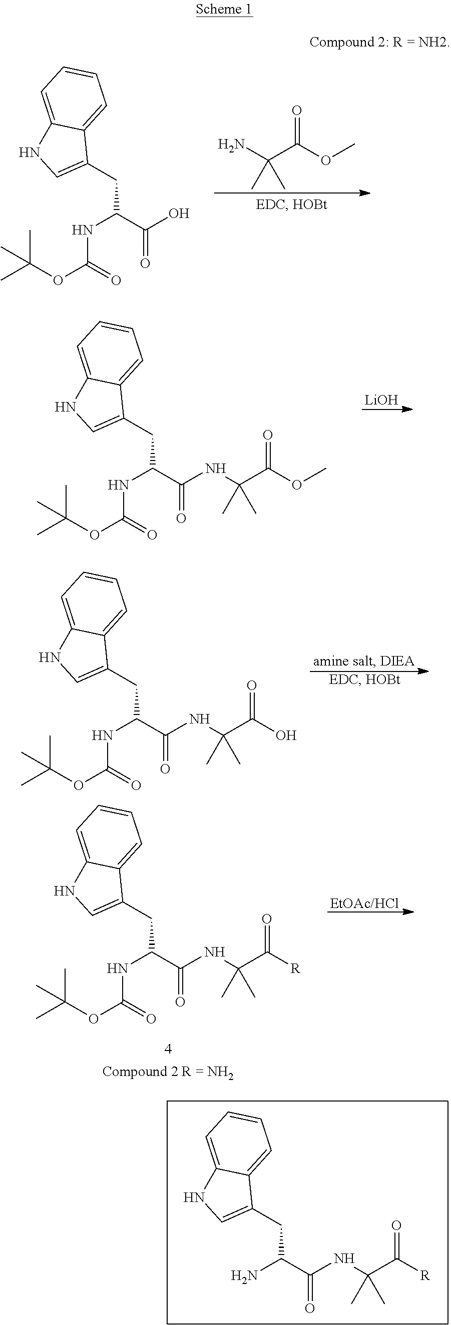

In addition, International Application publication WO 2012/055945 describes some embodiments of the synthesis of Compound 3 and Compound 4. In WO 2012/055945, Compound 3 described herein, is term compound “171”. (See WO 2012/055945 at Example 2 “Synthesis of compound (171)”). Briefly, Compound 3 was prepared according to the following steps:

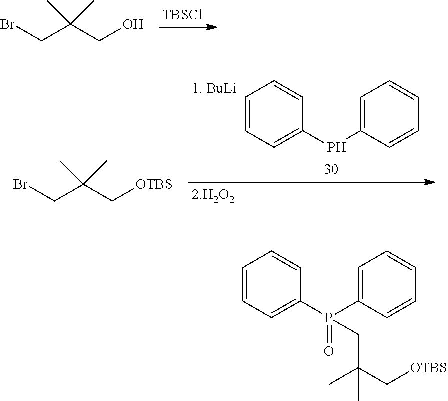

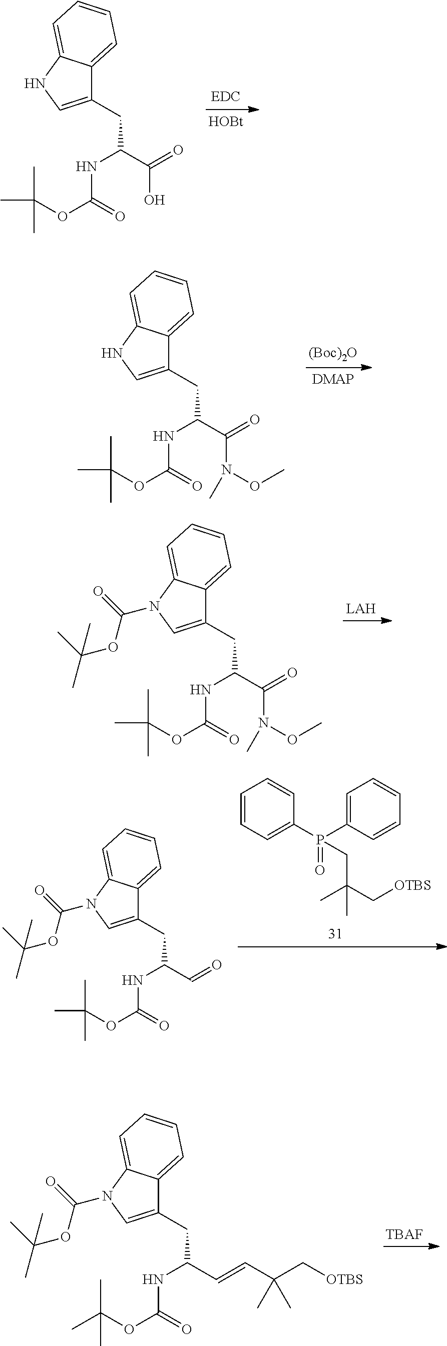

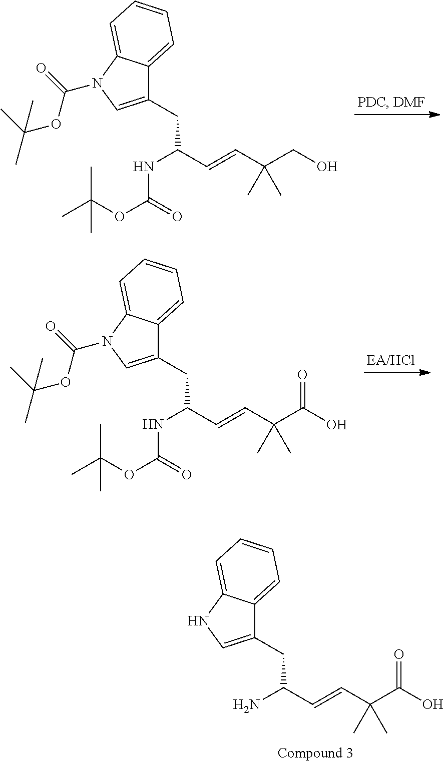

In WO 2012/055945, Compound 4 described herein, is term compound “121”. (See WO 2012/055945 at Example 1 “Synthesis of compound (121)”). Briefly, Compound 4 was prepared according to the following steps:

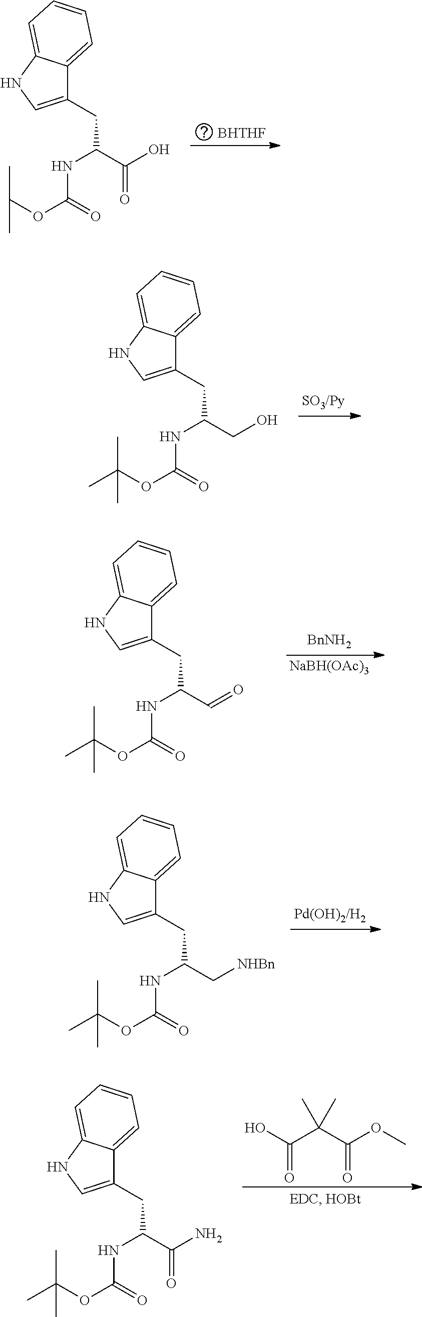

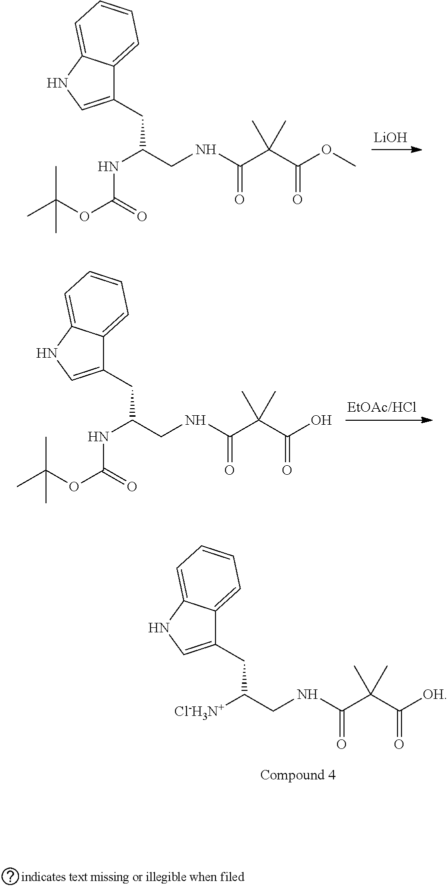

In some embodiments, a compound of Formula (I), (IA), (II) or (IIA) for use in the methods disclosed herein provides the active ingredient. In some embodiments, compound 1, 2, 3, 4, 5, 6, 7, 8, 9, 10, 11, 12, 13, 14, 15, 16, 17, 18, 19, 20, 21, 22, 23, 24, or 25, for use in the methods disclosed herein provides the active ingredient.

A skilled artisan would appreciate that the terms “pharmaceutically active agent” or “active agent” or “active pharmaceutical ingredient” or “active ingredient” are interchangeable and encompass the ingredient is a pharmaceutical drug which is biological active.

Preparation of Non-Toxic, Non-β-Sheet, Amorphous Aβ Clusters

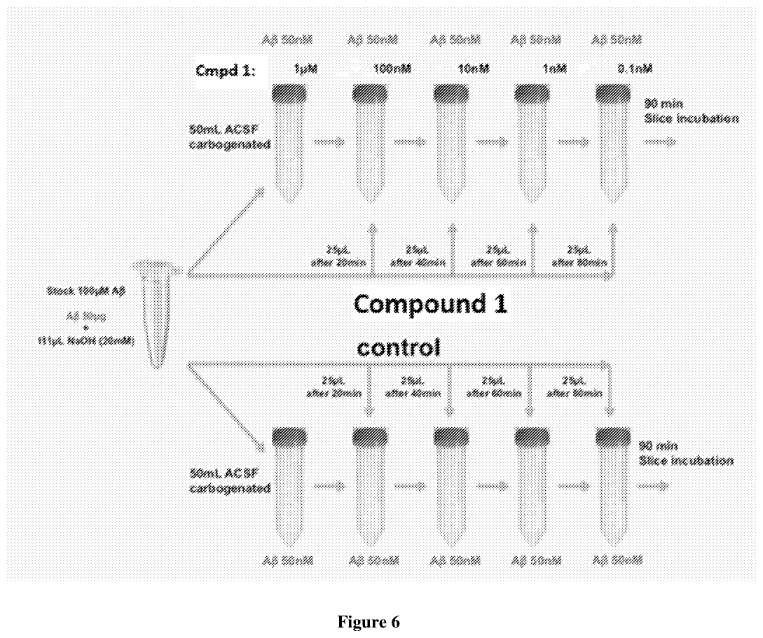

Methods of preparing a non-toxic, non-β-sheet, amorphous Aβ cluster comprising Aβ₁₋₄₂ and a compound of Formula I are described in detail below in and shown schematically in FIG. 6.

In some embodiments, a non-toxic, non-β-sheet, amorphous Aβ cluster comprises Aβ₁₋₄₂ and a compound of Formula I, or an optical isomer, pharmaceutically acceptable salt, hydrate, solvate, or polymorph thereof. In some embodiments, a non-toxic, non-β-sheet, amorphous Aβ cluster comprises Aβ₁₋₄₂ and a compound of Formula IA, or an optical isomer, pharmaceutically acceptable salt, hydrate, solvate, or polymorph thereof. In some embodiments, a non-toxic, non-β-sheet, amorphous Aβ cluster comprises Aβ₁₋₄₂ and a compound of Formula II, or an optical isomer, pharmaceutically acceptable salt, hydrate, solvate, or polymorph thereof. In some embodiments, a non-toxic, non-β-sheet, amorphous Aβ cluster comprises Aβ₁₋₄₂ and a compound of Formula IIA, or an optical isomer, pharmaceutically acceptable salt, hydrate, solvate, or polymorph thereof. In some embodiments, a non-toxic, non-β-sheet, amorphous Aβ cluster comprises Aβ₁₋₄₂ and any one of Compounds 1-25, or a pharmaceutically acceptable salt, hydrate, solvate, or polymorph thereof. In some embodiments, a non-toxic, non-β-sheet, amorphous Aβ cluster comprises Aβ₁₋₄₂ and Compound 1, or a pharmaceutically acceptable salt, hydrate, solvate, or polymorph thereof. In some embodiments, a non-toxic, non-β-sheet, amorphous Aβ cluster comprises Aβ₁₋₄₂ and Compound 2, or a pharmaceutically acceptable salt, hydrate, solvate, or polymorph thereof. In some embodiments, a non-toxic, non-β-sheet, amorphous Aβ cluster comprises Aβ₁₋₄₂ and Compound 3, or a pharmaceutically acceptable salt, hydrate, solvate, or polymorph thereof. In some embodiments, a non-toxic, non-β-sheet, amorphous Aβ cluster comprises Aβ₁₋₄₂ and Compound 4, or a pharmaceutically acceptable salt, hydrate, solvate, or polymorph thereof.

In some embodiments, a non-toxic, non-β-sheet, amorphous Aβ cluster comprises Aβ₁₋₄₂ and essentially no compound of Formula I, or no optical isomer, pharmaceutically acceptable salt, hydrate, solvate, or polymorph thereof.

In some embodiments, a compound of Formula I, or an optical isomer, a pharmaceutically acceptable salt, hydrate, solvate, or polymorph thereof is mixed with Aβ₁₋₄₂ and serially diluted, wherein the concentration of Aβ₁₋₄₂ is maintained and the concentration of the compound of Formula I is reduced. In some embodiments, a compound of Formula IA, or an optical isomer, a pharmaceutically acceptable salt, hydrate, solvate, or polymorph thereof is mixed with Aβ₁₋₄₂ and serially diluted, wherein the concentration of Aβ₁₋₄₂ is maintained and the concentration of the compound of Formula IA is reduced. In some embodiments, a compound of Formula II, or an optical isomer, a pharmaceutically acceptable salt, hydrate, solvate, or polymorph thereof is mixed with Aβ₁₋₄₂ and serially diluted, wherein the concentration of Aβ₁₋₄₂ is maintained and the concentration of the compound of Formula II is reduced. In some embodiments, a compound of Formula IIA or an optical isomer, a pharmaceutically acceptable salt, hydrate, solvate, or polymorph thereof is mixed with Aβ₁₋₄₂ and serially diluted, wherein the concentration of Aβ₁₋₄₂ is maintained and the concentration of the compound of Formula IIA is reduced. In some embodiments, a compound comprising any of compound 1-25, or a pharmaceutically acceptable salt, hydrate, solvate, or polymorph thereof, is mixed with Aβ₁₋₄₂ and serially diluted, wherein the concentration of Aβ₁₋₄₂ is maintained and the concentration of the compound comprising any of compound 1-25 is reduced.

In some embodiments, the series of dilutions starts with a 20:1 stoichiometric excess of a compound of Formula I, or an optical isomer, a pharmaceutically acceptable salt, hydrate, solvate, or polymorph thereof to Aβ₁₋₄₂. In some embodiments, the series of dilutions starts with about a 20:1 stoichiometric excess of a compound of Formula I, or an optical isomer, a pharmaceutically acceptable salt, hydrate, solvate, or polymorph thereof to Aβ₁₋₄₂. In some embodiments, the series of dilutions starts with an about 30:1 to 20:1 stoichiometric excess of a compound of Formula I, or an optical isomer, a pharmaceutically acceptable salt, hydrate, solvate, or polymorph thereof to Aβ₁₋₄₂. In some embodiments, the series of dilutions starts with an about 20:1 to 10:1 stoichiometric excess of a compound of Formula I, or an optical isomer, a pharmaceutically acceptable salt, hydrate, solvate, or polymorph thereof to Aβ₁₋₄₂.

In some embodiments, the series of dilutions comprises about 2-10 dilution steps. In some embodiments, the series of dilutions comprises about 3-10 dilution steps. In some embodiments, the series of dilutions comprises about 4-10 dilution steps. In some embodiments, the series of dilutions comprises about 5-10 dilution steps. In some embodiments, the series of dilutions comprises about 2-5 dilution steps. In some embodiments, the series of dilutions comprises about 3-5 dilution steps. In some embodiments, the series of dilutions comprises about 4-5 dilution steps. In some embodiments, the series of dilutions comprises 2 dilution steps. In some embodiments, the series of dilutions comprises 3 dilution steps. In some embodiments, the series of dilutions comprises 4 dilution steps. In some embodiments, the series of dilutions comprises 5 dilution steps. In some embodiments, the series of dilutions comprises 6 dilution steps. In some embodiments, the series of dilutions comprises 7 dilution steps. In some embodiments, the series of dilutions comprises 8 dilution steps. In some embodiments, the series of dilutions comprises 9 dilution steps. In some embodiments, the series of dilutions comprises 10 dilution steps.

In some embodiments, the starting concentration of a compound of Formula I, or an optical isomer, a pharmaceutically acceptable salt, hydrate, solvate, or polymorph thereof, is 1 μM and the maintained concentration of Aβ₁₋₄₂ is 50 nM, wherein the dilution series start with a 20:1 stoichiometric excess to Aβ₁₋₄₂, and there are 5 dilution steps.

In some embodiments, the final dilution mixture that in some embodiments would be used in a method of reversing Aβ toxicity and rapidly improving function of neuronal cells, non-neuronal cells, or neurosensory cells, or a combination thereof, comprises a 500:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture that in some embodiments would be used in a method of reversing Aβ toxicity and rapidly improving function of neuronal cells, non-neuronal cells, or neurosensory cells, or a combination thereof, comprises between a 250:1 to 500:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises between a 250:1 to 1000:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises between a 250:1 to 500:1 stoichiometric excess of Aβ₁₋₄₂ to the compound of formula I, or an optical isomer, a pharmaceutically acceptable salt, hydrate, solvate, or polymorph thereof.

In some embodiments, the final dilution mixture comprises a 250:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 300:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 350:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 400:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 450:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 500:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 550:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a 650:1, a 700:1, a 750:1, a 800:1, a 850:1, a 900:1, a 950:1, or a 1000:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises greater than a 250:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises greater than a 500:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises greater than a 1000:1 stoichiometric excess of Aβ₁₋₄₂ to the compound of formula I, or an optical isomer, a pharmaceutically acceptable salt, hydrate, solvate, or polymorph thereof. In some embodiments, the final dilution mixture comprises a negligible concentration of a compound of formula I, or an optical isomer, a pharmaceutically acceptable salt, hydrate, solvate, or polymorph thereof. In some embodiments, the final dilution mixture comprises a negligible quantity of a compound of formula I, or an optical isomer, a pharmaceutically acceptable salt, hydrate, solvate, or polymorph thereof.

In some embodiments, the final concentration of a compound of Formula I, or an optical isomer, a pharmaceutically acceptable salt, hydrate, solvate, or polymorph thereof, comprised in an amorphous cluster is about 0.1 nM. In some embodiments, the final concentration of a compound of Formula I, or an optical isomer, a pharmaceutically acceptable salt, hydrate, solvate, or polymorph thereof, comprised in an amorphous cluster is about 0.5 nM. In some embodiments, the final concentration of a compound of Formula I, or an optical isomer, a pharmaceutically acceptable salt, hydrate, solvate, or polymorph thereof, comprised in an amorphous cluster is between about 0.5 nM-0.05 nM. In some embodiments, the final concentration of a compound of Formula I, or an optical isomer, a pharmaceutically acceptable salt, hydrate, solvate, or polymorph thereof, comprised in an amorphous cluster is between about 0.1 nM-0.01 nM. In some embodiments, the final concentration of a compound of Formula I, or an optical isomer, a pharmaceutically acceptable salt, hydrate, solvate, or polymorph thereof, comprised in an amorphous cluster is less than 0.5 nM. In some embodiments, the final concentration of a compound of Formula I, or an optical isomer, a pharmaceutically acceptable salt, hydrate, solvate, or polymorph thereof, comprised in an amorphous cluster is less than 0.1 nM. In some embodiments, the final concentration of a compound of Formula I, or an optical isomer, a pharmaceutically acceptable salt, hydrate, solvate, or polymorph thereof, comprised in an amorphous cluster is less than 0.05 nM. In some embodiments, the final concentration of a compound of Formula I, or an optical isomer, a pharmaceutically acceptable salt, hydrate, solvate, or polymorph thereof, comprised in an amorphous cluster is less than 0.01 nM. In some embodiments, the final concentration of a compound of Formula I, or an optical isomer, a pharmaceutically acceptable salt, hydrate, solvate, or polymorph thereof, comprised in an amorphous cluster is less than 0.005 nM. In some embodiments, the final concentration of a compound of Formula I, or an optical isomer, a pharmaceutically acceptable salt, hydrate, solvate, or polymorph thereof, comprised in an amorphous cluster is less than 0.001 nM. In some embodiments, the final concentration of a compound of Formula I, or an optical isomer, a pharmaceutically acceptable salt, hydrate, solvate, or polymorph thereof, comprised in an amorphous cluster is negligible.

In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising between a compound of Formula I, or an optical isomer, a pharmaceutically acceptable salt, hydrate, solvate, or polymorph thereof at about 0.5 nM-0.05 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.1 nM-0.01 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.005 nM-0.0005 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.001 nM-0.0001 nM.

In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.5 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.1 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.05 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.01 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.005 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at about 0.001 nM. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising a compound of Formula I, or an optical isomer, a pharmaceutically acceptable salt, hydrate, solvate, or polymorph thereof at a negligible concentration. In some embodiments, a method disclosed herein to reverse Aβ toxicity and rapidly improved function of neuronal, non-neuronal, or neurosensory cells, or a combination thereof, comprising administering non-toxic, non-β-sheet, amorphous Aβ clusters comprising an absence of a compound of Formula I, or an optical isomer, a pharmaceutically acceptable salt, hydrate, solvate, or polymorph thereof.

In some embodiments, use of a non-toxic, non-β-sheet, amorphous Aβ cluster as described herein, detoxifies misfolded amyloid β monomers. In some embodiments, use of a non-toxic, non-β-sheet, amorphous Aβ cluster as described herein, detoxifies misfolded amyloid β oligomers.

In some embodiments, the non-toxic, non-β-sheet, amorphous Aβ cluster is comprised in a pharmaceutically acceptable composition.

Compositions

In one embodiment, a “pharmaceutical composition” refers to a preparation of one or more of the active ingredients described herein with other chemical components such as physiologically suitable carriers and excipients. The purpose of a pharmaceutical composition is to facilitate administration of a compound to an organism. In certain embodiments, a “pharmaceutical composition” provides the pharmaceutical dosage form of a drug. “Pharmaceutical compositions” in certain embodiments include any known dosage form in the art. As used herein, the terms “pharmaceutical composition” or “composition” or “formulation” may be used interchangeably having all the same meanings and qualities.

A skilled artisan would appreciate that the phrase “pharmaceutically acceptable”, as used in connection with compositions described herein, refers to molecular entities and other ingredients of such compositions which are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

The active ingredients, for example, the compound of Formula (I), (IA), (II) or (IIA), for example but not limited to any of the compounds 1-25, for use in the methods disclosed herein, together with one or more conventional excipients (adjuvants, carriers, or diluents) may be placed into the form of pharmaceutical compositions and unit dosages thereof. In some embodiments, a pharmaceutical composition described herein comprises a sterile formulation. In some embodiments, a pharmaceutical composition described herein comprises an excipient.

The compositions may be employed as solids, such as coated or uncoated tablets or filled capsules; or liquids, such as solutions, suspensions, emulsions, or capsules filled with the same; or may be employed as aerosols, such as a spray or mists. The compositions can be prepared for oral use. They can be in the form of suppositories or capsules for rectal administration. In some embodiments, compositions are prepared for nasal use, for example a nasal spray or mist. In some embodiments, compositions are prepared for use in the eye in the form of eye-drops or as a sterile injectable solution for intra-ocular administering. In some embodiments, compositions are prepared for systemic use in the form of an injectable solution, for example but not limited to, for intrathecal, subcutaneous, implanted slow-release depots, direct injection using an in-dwelling catheter, intramuscular, or intravenous injection. In some embodiments, compositions are prepared for systemic or local use in the form of a topical ointment, a patch, or a dermal patch.

Compositions can be in the form of sterile injectable solutions for parenteral (including intrathecal, subcutaneous, intramuscular, direct injection using an in-dwelling catheter, implanted slow release depots, or intravenous injection) use. They can be in liquid or semi-liquid form for ophthalmic application to the eye (including eye-drops or intra-ocular injection). In some embodiments, ophthalmic application to the eye uses a composition in the form of eye drops, eye creams, and intraocular depot formulations. In some embodiments, compositions are in the form of nose sprays or mists for treatment of ophthalmic conditions. In some embodiments, compositions are in the form of nose sprays or mists for treatment of neurological conditions.

Such pharmaceutical compositions and unit dosage forms thereof may comprise conventional or new ingredients in conventional or special proportions, with or without additional active compounds. Such unit dosage forms may contain any suitable effective amount of the active ingredient of Formula (I), (IA), (II) or (IIA) commensurate with the intended dosage range to be employed. In some embodiments, unit dosage forms may contain any suitable effective amount of the active ingredient of any one of compounds 1-25 commensurate with the intended dosage range to be employed. In some embodiments, unit dosage forms may contain any suitable effective amount of the active ingredient of compound 1, 2, 3, or 4, commensurate with the intended dosage range to be employed.

In some embodiments, compositions containing 0.5 to 1000 milligrams, preferably 1 to 100 milligrams of active ingredient per application unit are suitable representative unit dosage forms. In some embodiments, compositions containing about 0.01-10 mg/kg bodyweight on peroral administration and 0.001-10 mg/kg bodyweight on parenteral administration.

In one embodiment, as used herein, the term “excipient” applied to pharmaceutical compositions for the method disclosed herein refers to a diluent, adjuvant, or carrier with which an active compound of Formula (I), (IA), (II) or (IIA) or of any one of compounds 1-25 is administered. Such pharmaceutical excipients often are sterile liquids, such as water or saline solutions. Other excipients, depending on the type of administration, can be aqueous dextrose solutions, aqueous glycerol solutions, and oils, including those of animal, vegetable or synthetic origin (see Remington and AR. Gennaro, 20th Edition, (2000) “Remington: The Science and Practice of Pharmacy”, published by Lippincott, Williams, and Wilkins.). In some embodiments, a pharmaceutical composition comprising an active compound of Formula (I), (IA), (II) or (IIA) or of any one of compounds 1-25, comprises the excipient cyclodextrin.

For ophthalmological applications (for ocular diseases and disorders), topic formulations are often applied. They are often water-based solutions or dispersions. However, water-free solutions or suspensions could also be used.

The compound of Formula (I), (IA), (II) or (IIA), or any of compounds 1-25 can also be administered orally in the form of a capsule, a tablet, or the like. The orally administered compositions can be administered in the form of a time-controlled release vehicle, including diffusion-controlled systems, osmotic devices, dissolution-controlled matrices, and erodible/degradable matrices.

For oral administration in the form of a tablet or capsule, the compound of Formula (I) or (IA) or (II) or (IIA) or any of compounds 1-25, for example but not limited to compounds 1, 2, 3, or 4, may be combined with non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. The tablets containing compound of Formula (I) or (IA) or (II) or (IIA) or any of compounds 1-25, for example but not limited to compounds 1, 2, 3, or 4 may be coated by methods well known in the art.

For oral administration in liquid form, the drug components may be combined with non-toxic, pharmaceutically acceptable inert carriers or solvents (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-phydroxybenzoates or sorbic acid), and the like. Stabilizing agents such as antioxidants (BRA, BRT, propyl gallate, sodium ascorbate, citric acid) may also be added to stabilize the dosage forms.

The compositions for the method disclosed herein containing a compound of Formula (I) or (IA) or (II) or (IIA) or any of compounds 1-25, for example but not limited to compounds 1, 2, 3, or 4 may be also introduced in beads, microspheres or microcapsules, e.g., fabricated from polyglycolic acid/lactic acid (PGLA). Liquid preparations for oral administration may take the form of solutions, syrups, emulsions or suspensions, or they may be presented as a dry product for reconstitution with water or other suitable vehicle before use. Preparations for oral administration may be suitably formulated to give controlled or postponed release of the active compound.

The active drugs of Formula (I), (IA), (II) or (IIA), or any of compounds 1-25, for example but not limited to compounds 1, 2, 3, or 4, may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines, as is well known.

The active compound of Formula (I), (IA), (II) or (IIA), or any of compounds 1-25, for example but not limited to compounds 1, 2, 3, or 4, may also be coupled with soluble polymers as targetable drug carriers. Such polymers include polyvinyl-pyrrolidone, pyran copolymer, polyhydroxypropyl methacrylamide-phenol, polyhydroxy-ethyl-aspartamide-phenol, or polyethyleneoxidepolylysine substituted with palmitoyl residues. Furthermore, the compound of Formula (I), (IA), (II) or (IIA) may be coupled to a class of biodegradable polymers useful in achieving controlled release of a drug, for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

For administration by inhalation, the therapeutics according to the methods described herein using as an active compound a compound of Formula (I), (IA), (II) or (IIA), or any of compounds 1-25, for example but not limited to compounds 1, 2, 3, or 4, may be conveniently delivered in the form of an aerosol spray presentation from pressurized packs or a nebulizer, with the use of a suitable propellant, e.g. dichlorodifluoromethane or other suitable gas.

For administration by aerosol spray (for example but not limited to a nasal spray or mist), the therapeutics according to the methods of use containing as active compound, which in some embodiments comprises a compound of Formula (I), (IA), (II) or (IIA), or any of compounds 1-25, for example but not limited to compounds 1, 2, 3, or 4, may be conveniently delivered in the form of an aerosol spray or mist from pressurized packs or a nebulizer, with the use of a suitable propellant, e.g. dichlorodifluoromethane or other suitable gas.

The formulations for use in the methods disclosed herein containing a compound of formula (I), (IA), (II) or (IIA), or any of compounds 1-25, for example but not limited to compounds 1, 2, 3, or 4, may be delivered parenterally, i.e., by intravenous (i.v.), intracerebroventricular (i.c.v.), subcutaneous (s.c.), intraperitoneal (i.p.), intramuscular (i.m.), subdermal (s.d.), intrathecal (i.th.), intraocular (intravitreal), periocular, implanted slow-release depots, direct injection using an in-dwelling catheter, or intradermal (i.d.) administration, by direct injection, e.g. via bolus injection or continuous infusion.

Formulations for use in the methods disclosed herein containing a compound of formula (I), (II), (IIA), or any of compounds 1-25, for example but not limited to compounds 1, 2, 3, or 4, for injection, (intraocular injection in particular for application to the eye) can be presented in unit dosage form, e.g., in ampoules or in multi-dose containers, with an added preservative. The compositions can be a suspension, solutions, or emulsion e.g. in aqueous vehicles, and can contain excipients such as suspending, stabilizing and/or dispersing agents. Alternatively, the compound of formula (I), (IA), (II) or (IIA), or any of compounds 1-25, for example but not limited to compounds 1, 2, 3, or 4, can be in powder form for reconstitution with a suitable excipient, e.g., sterile pyrogen-free water, for reconstitution.

Formulations for use in the methods disclosed herein containing a compound of Formula (I), (II), (IIA), or any of compounds 1-25, for example but not limited to compounds 1, 2, 3, or 4, for injection, can be presented in unit dosage form, e.g., in ampoules or in multi-dose containers, with an added preservative. The compositions can be a suspension, solutions, or emulsion e.g. in aqueous vehicles, and can contain excipients such as suspending, stabilizing and/or dispersing agents. Alternatively, the compound of formula (I), (IA), (II) or (IIA), or any of compounds 1-25, for example but not limited to compounds 1, 2, 3, or 4, can be in powder form for reconstitution with a suitable excipient, e.g., sterile pyrogen-free water, for reconstitution.

Compositions for the method of use of a composition containing a compound of Formula (I) (II), (IIA), or any of compounds 1-25, for example but not limited to compounds 1, 2, 3, or 4, may also be formulated for rectal administration, e.g., as suppositories or retention enemas (e.g., containing conventional suppository bases such as cocoa butter or other glycerides).

The compositions containing a compound of formula (I), (II), (IIA), or any of compounds 1-25, for example but not limited to compounds 1, 2, 3, or 4, may be presented in a pack or dispenser device, which may contain one or more unit dosage forms containing the active ingredient and/or may contain different dosage levels to facilitate dosage titration. The pack may comprise metal or plastic foil, such as a blister pack. The pack or dispenser device may be accompanied by instructions for administration. Compositions for the method disclosed herein formulated in a compatible pharmaceutical carrier may also be prepared, placed in an appropriate container, and labeled for treatment of an indicated condition.

As disclosed herein, the dose of the components in the compositions for the method of use disclosed herein is determined to ensure that the dose administered continuously or intermittently will not exceed an amount determined after consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, seriousness of the disease. The appropriate dose and dosing times under certain conditions can be determined by the test based on the above-described indices but may be refined and ultimately decided according to the judgment of the practitioner and each patient's circumstances (age, general condition, severity of symptoms, sex, etc.) according to standard clinical techniques.

Toxicity and therapeutic efficacy of the compositions for the method disclosed herein can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it may be expressed as the ratio ED₅₀/LD₅₀. Those pharmaceutical compositions that exhibit large therapeutic indices are preferred.

In some embodiments, each dose used in a method described herein comprises 100% of the therapeutically effective dose. In some embodiments, each dose used in a method described herein comprises 20-75% of the therapeutically effective dose. In some embodiments, each dose used in a method described herein comprises 20, 25, 30, 35, 40, 45, 50, 55, 60, 65, 70, or 75% of the therapeutically effective dose.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments described may be presented in a range format. It should be understood that the description in range format is merely for convenience and brevity and should not be construed as an inflexible limitation on the scope of for example, but not limited to percent of a therapeutically effective dose. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “about” refers to plus/minus 10% of the value stated.

In some embodiments, individual doses of multiple doses to be administered each comprise 100% of the therapeutically effective dose, or 75-100% of the therapeutically effective dose, or 20-75% of the therapeutically effective dose, or any combination thereof.

In some embodiments, methods of use described herein administer a compound of formula (I), (IA), (II), or (IIA), or an optical isomer, a pharmaceutically acceptable salt, a hydrate, a solvate, or a polymorph thereof, wherein the compound is comprised in a pharmaceutically acceptable composition. In some embodiments, methods of use described herein administer any of compound 1-25 or a pharmaceutically acceptable salt, a hydrate, a solvate, or a polymorph thereof, wherein the compound is comprised in a pharmaceutically acceptable composition. In some embodiments, methods of use described herein administer any of compound 1, 2, 3, or 4, or a pharmaceutically acceptable salt, a hydrate, a solvate, or a polymorph thereof, wherein the compound is comprised in a pharmaceutically acceptable composition. In some embodiments, methods of use described herein administer compound 1 or a pharmaceutically acceptable salt, a hydrate, a solvate, or a polymorph thereof, wherein the compound is comprised in a pharmaceutically acceptable composition. In some embodiments, methods of use described herein administer compound 2 or a pharmaceutically acceptable salt, a hydrate, a solvate, or a polymorph thereof, wherein the compound is comprised in a pharmaceutically acceptable composition. In some embodiments, methods of use described herein administer compound 3 or a pharmaceutically acceptable salt, a hydrate, a solvate, or a polymorph thereof, wherein the compound is comprised in a pharmaceutically acceptable composition. In some embodiments, methods of use described herein administer compound 4 or a pharmaceutically acceptable salt, a hydrate, a solvate, or a polymorph thereof, wherein the compound is comprised in a pharmaceutically acceptable composition. In some embodiments, methods of use described herein administer non-toxic, non-β sheet, amorphous Aβ clusters comprised in a pharmaceutically acceptable composition.

Methods of Use

Misfolded Amyloid β1-42 (Aβ₁₋₄₂) is a major endogenous pathogen underlying the etiology of amyloid β diseases and conditions. Misfolded Aβ₁₋₄₂ monomers may bind to each other forming toxic soluble Aβ oligomers, which cause synaptic dysfunction and neurodegeneration in amyloid β diseases and conditions. These toxic Aβ₁₋₄₂ oligomers may damage, reduce functionality, inhibit functionality, or alter functionality of neuronal, non-neuronal, and/or sensory cells affected in amyloid β diseases and conditions.

In some embodiments, a method to reverse amyloid β functional toxicity of neuronal, non-neuronal, and neuro-sensory cells in a subject in need, comprises administration of a pharmaceutically effective amount of Compound of Formula I

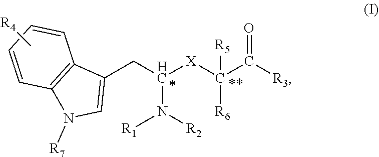

wherein
* refers to a chiral center;
* * refers to a chiral center if R₅ and R₆ are different;
R₁ is hydrogen, —C₁₋₆-alkyl, cycloC₃₋₁₂-alkyl, —C(O)R or —C(O)OR;
R₂ is hydrogen, C₁₋₆-alkyl, or cycloC₃₋₁₂-alkyl;
R₃ is —OR, —NHR or —N(R)₂;
R₄ is hydrogen, halogen, cyano, trifluoromethyl, —C₁₋₆-alkyl, —C₆₋₁₀-aryl, heteroaryl, —OR, —NHR, —N(R)₂, —C(O)R or —C(O)—NHR;
R⁵ is hydrogen, —C₁₋₆-alkyl or C₂₋₆-alkenyl; or
R⁵ and R⁶ together with the carbon atom carrying them form a cyclic system with 3 to 6 carbon atoms;
R⁶ is hydrogen, —C₁₋₆-alkyl or C₂₋₆-alkenyl;
R⁷ is hydrogen, methyl, ethyl, propyl or cyclopropyl;
R is hydrogen, —C₁₋₆-alkyl, or —C₆₋₁₀-aryl; and
X is a group —C(O)CH₂—, —CH(OH)CH₂—, —CH═CH—, —CH₂—NR—C(O)—, or —C(O)NR;
or an optical isomer, a pharmaceutically acceptable salt, a hydrate, a solvate, or a polymorph thereof.

In some embodiments, a method to reverse amyloid β functional toxicity of neuronal, non-neuronal, or neuro-sensory cells, or a combination thereof in a subject in need, comprises administration of a pharmaceutically effective amount of a non-toxic, non-β-sheet, amorphous Aβ cluster comprising a Compound of Formula I, or an optical isomer, a pharmaceutically acceptable salt, a hydrate, a solvate, or a polymorph thereof. In some embodiments, a method to reverse amyloid β functional toxicity of neuronal, non-neuronal, or neuro-sensory cells, or a combination thereof in a subject in need, comprises administration of a pharmaceutically effective amount of a non-toxic, non-β-sheet, amorphous Aβ cluster not comprising a Compound of Formula I, or an optical isomer, a pharmaceutically acceptable salt, a hydrate, a solvate, or a polymorph thereof.

In some embodiments, a method to reverse amyloid β toxicity and rapidly improve the function of neuronal, non-neuronal, or neuro-sensory cells, or a combination thereof in a subject in need, comprises administration of a pharmaceutically effective amount of Compound of Formula I, or an optical isomer, a pharmaceutically acceptable salt, a hydrate, a solvate, or a polymorph thereof. In some embodiments, a method to reverse amyloid β toxicity and rapidly improve the function of neuronal, non-neuronal, or neuro-sensory cells, or a combination thereof in a subject in need, comprises administration of a pharmaceutically effective amount of a non-toxic, non-β-sheet, amorphous Aβ cluster comprising a Compound of Formula I, or an optical isomer, a pharmaceutically acceptable salt, a hydrate, a solvate, or a polymorph thereof. In some embodiments, a method to reverse amyloid β toxicity and rapidly improve the function of neuronal, non-neuronal, or neuro-sensory cells, or a combination thereof in a subject in need, comprises administration of a pharmaceutically effective amount of a non-toxic, non-β-sheet, amorphous Aβ cluster not comprising a Compound of Formula I, or an optical isomer, a pharmaceutically acceptable salt, a hydrate, a solvate, or a polymorph thereof.

Detailed embodiments of compounds of Formula I are provided above. Embodiments of the compounds of Formula I provided above, are incorporated herein in their entirety. As well detailed embodiments of non-toxic, non-β-sheet, amorphous Aβ clusters are provided above. Embodiments of the non-toxic, non-β-sheet, amorphous Aβ clusters provided above, are incorporated herein in their entirety.

In some embodiments, compounds of Formulae (I), (IA), (II), or (IIA), reverse Aβ functional toxicity. In some embodiments, compounds of Formulae (I), (IA), (II), or (IIA), reverse Aβ functional toxicity in vivo. In some embodiments, compounds of Formulae (I), (IA), (II), or (IIA) reverse amyloid β functional toxicity on neuronal cells, on non-neuronal cells, or on neuro-sensory cells. In some embodiments, compounds of Formulae (I), (IA), (II), or (IIA) reverse AR functional toxicity on neuronal cells in the central nervous system such as, but not exclusively, pyramidal and other excitatory neurons in the hippocampus and cortex. In some embodiments, compounds of Formulae (I), (IA), (II), or (IIA) reverse Aβ functional toxicity on retinal ganglion cells (RGC). In some embodiments, compounds of Formulae (I), (IA), (II), or (IIA) reverse amyloid β functional toxicity on retinal pigment epithelium cells (RPE).

In some embodiments, compounds of Formulae (I), (IA), (II), or (IIA) reverse amyloid 3 functional toxicity on photosensory cells comprises rod and cone cells. In some embodiments, compounds of Formulae (I), (IA), (II), or (IIA) reverse Aβ functional toxicity on hippocampal cells.

In some embodiments, neuronal cells comprise Hippocampal cells, Cortical Pyramidal cells, Inhibitory interneurons, Place cells, Basket cells, Granule cells, Retinal ganglion cells (RGC), Bipolar cells, Horizontal cells, and Amacrine cells. In some embodiments, non-neuronal cells comprise Retinal pigment epithelium (RPE) cells, Astrocytes, and Oligodendrocytes. In some embodiments, neuronal sensory cells comprise photosensory cells for example but not limited to rod cells and cone cells.

In some embodiments, disclosed herein is a method to reverse amyloid β functional toxicity of neuronal, non-neuronal, and neuro-sensory cells in a subject in need, said method comprising administration of a pharmaceutically effective amount of a Compound of Formula IA:

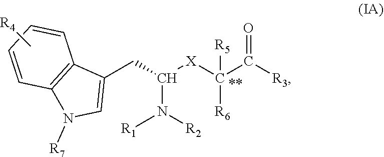

wherein variables R₁, R₂, R₃, R₄, R₅, R₆, R₇, and X are defined for the structure of formula I, or an optical isomer, a pharmaceutically acceptable salt, a hydrate, a solvate, or a polymorph thereof.

In some embodiments, disclosed herein is a method to reverse amyloid β functional toxicity of neuronal, non-neuronal, and neuro-sensory cells in a subject in need, said method comprising administration of a pharmaceutically effective amount of Compound 1:

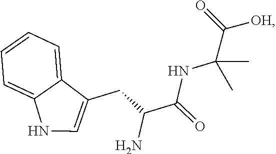

or a pharmaceutically acceptable salt, a hydrate, a solvate, or a polymorph thereof.

In some embodiments, disclosed herein is a method to reverse amyloid β functional toxicity of neuronal, non-neuronal, and neuro-sensory cells in a subject in need, said method comprising administration of a pharmaceutically effective amount of Compound 2:

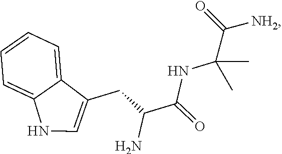

or a pharmaceutically acceptable salt, a hydrate, a solvate, or a polymorph thereof.

In some embodiments, disclosed herein is a method to reverse amyloid β functional toxicity of neuronal, non-neuronal, and neuro-sensory cells in a subject in need, said method comprising administration of a pharmaceutically effective amount of Compound 3:

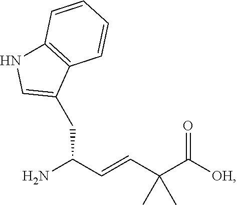 [SMILES: CC(C)(/C=C/[C@H](N)Cc1c[nH]c2ccccc12)C(=O)O]

or a pharmaceutically acceptable salt, a hydrate, a solvate, or a polymorph thereof.

In some embodiments, disclosed herein is a method to reverse amyloid β functional toxicity of neuronal, non-neuronal, and neuro-sensory cells in a subject in need, said method comprising administration of a pharmaceutically effective amount of Compound 4:

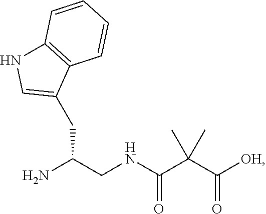 [SMILES: CC(C)(C(=O)O)C(=O)NC[C@H](N)Cc1c[nH]c2ccccc12]

or a pharmaceutically acceptable salt, a hydrate, a solvate, or a polymorph thereof.

In some embodiments, disclosed herein is a method to reverse amyloid β functional toxicity of neuronal, non-neuronal, and neuro-sensory cells in a subject in need, said method comprising administration of a pharmaceutically effective amount of a compound selected from Compounds 5-25:

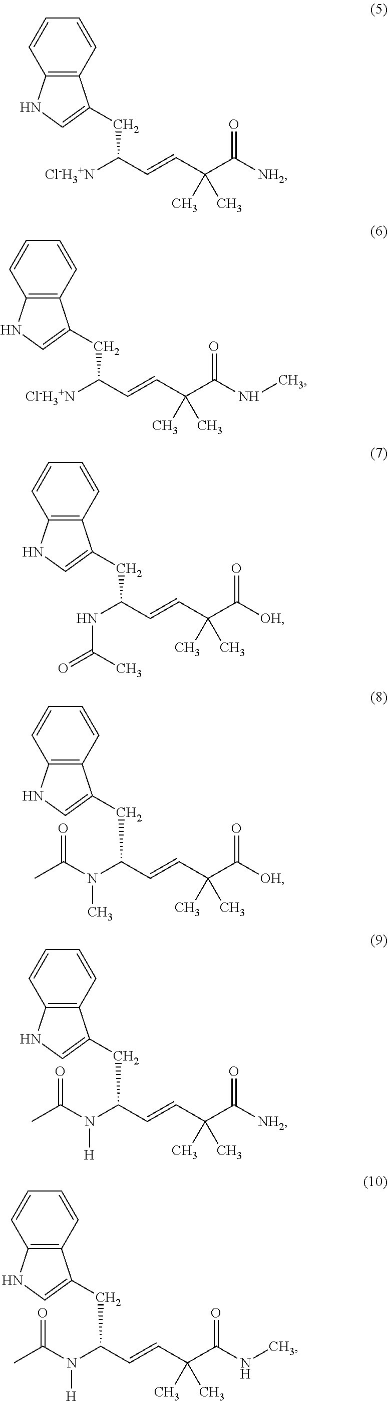

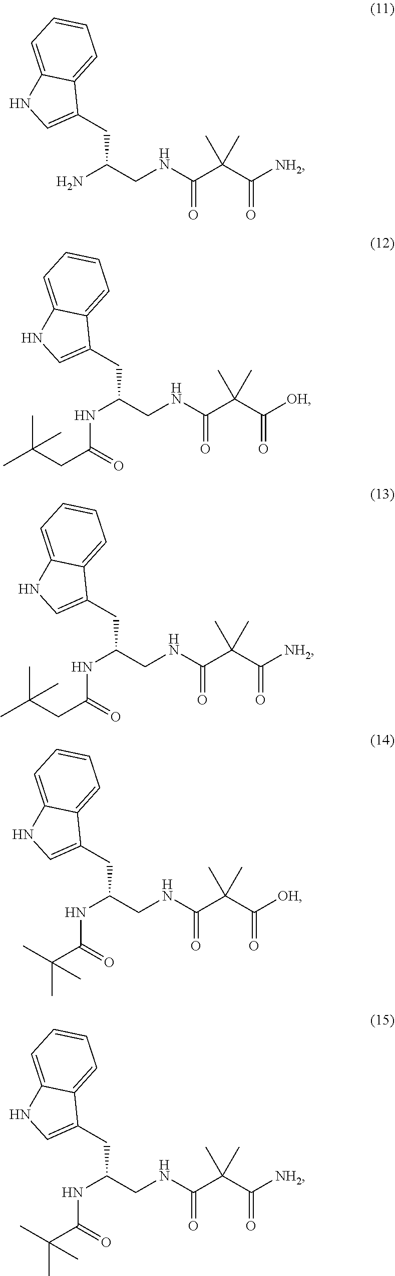

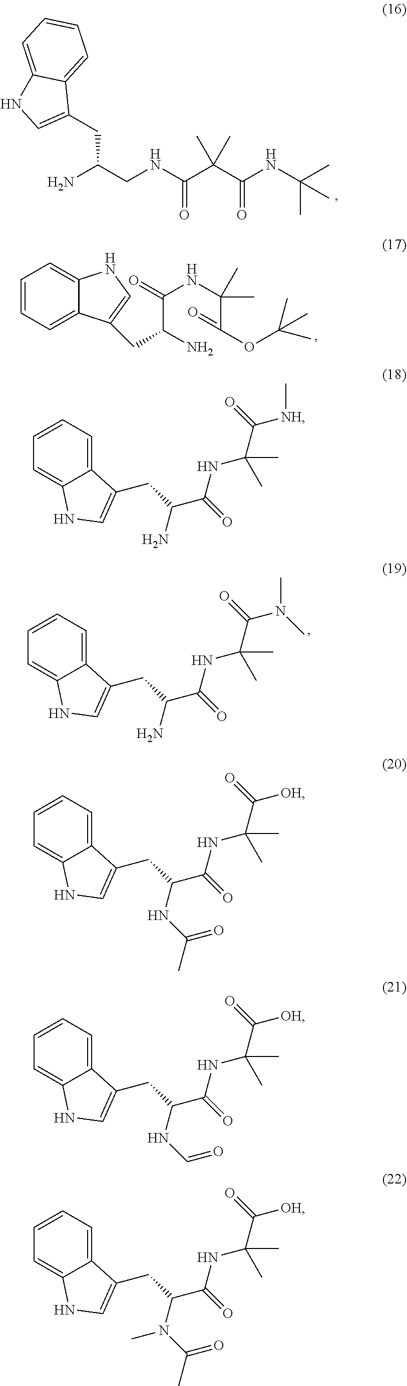

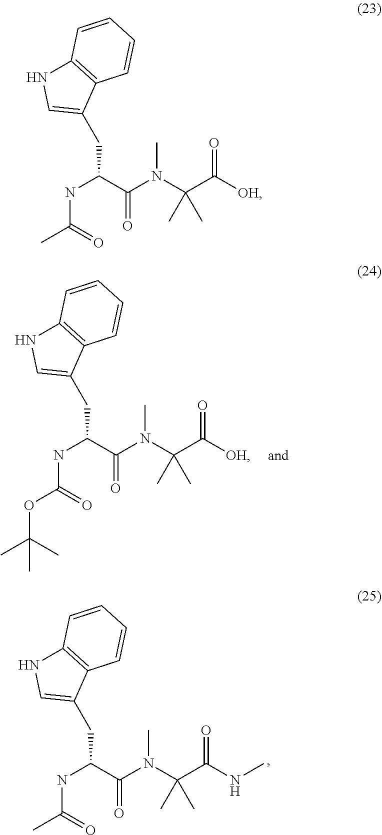

or a pharmaceutically acceptable salt, a hydrate, a solvate, or a polymorph thereof.

A skilled artisan would appreciate that neuronal cells include but are not limited to retinal ganglion cells (RGC), Hippocampal cells, Cortical Pyramidal cells, Inhibitory interneurons, Place cells, Basket cells, Granule cells, Bipolar cells, Horizontal cells, and Amacrine cells. The function of these cells may be damaged, reduced, inhibited, or altered in a subject suffering from an amyloid β-associated disease or condition. In some embodiments, neuronal cells comprise RGC.

A skilled artisan would appreciate that non-neuronal cells may encompass retinal pigment epithelial (RPE) cells, Astrocytes, and Oligodendrocytes, astrocytes. The function of these cells may be damaged, reduced, inhibited, or altered in a subject suffering from an amyloid β-associated disease or condition. In some embodiments, non-neuronal cells comprise RPE cells.

A skilled artisan would appreciate that neuro-sensory cells may encompass neurons that convert a specific type of stimulus, via their receptors, into action potentials or graded potentials. Examples of neurosensory cells are the photosensory cells of the eye: rod cells and cone cells. The function of these cells may be damaged, reduced, inhibited, or altered in a subject suffering from an amyloid β-associated disease or condition. In some embodiments, neurosensory cells also comprise retinal ganglion cells (RGC), cone cells, and rod cells.

In some embodiments, any of Compound 1-25 reverses amyloid β functional toxicity. In some embodiments, any of Compound 1-25 reverses amyloid β functional toxicity in vivo. In some embodiments, any of Compound 1-25 reverses amyloid β functional toxicity on neuronal cells, on non-neuronal cells, or on neuro-sensory cells. In some embodiments, any of Compound 1-25 reverses amyloid β functional toxicity on retinal ganglion cells (RGC). In some embodiments, any of Compound 1-25 reverses amyloid β functional toxicity on retinal pigment epithelium cells (RPE). In some embodiments, any of Compound 1-25 reverses amyloid β functional toxicity on cone cells. In some embodiments, any of Compound 1-25 reverses amyloid β functional toxicity on rod cells.

In some embodiments, Compound 1 reverses amyloid β functional toxicity. In some embodiments, Compound 1 reverses amyloid β functional toxicity in vivo. In some embodiments, Compound 1 reverses amyloid β functional toxicity on neuronal cells, on non-neuronal cells, or on neuro-sensory cells. In some embodiments, Compound 1 reverses amyloid β functional toxicity on retinal ganglion cells (RGC). In some embodiments, Compound 1 reverses amyloid β functional toxicity on retinal pigment epithelium cells (RPE). In some embodiments, Compound 1 reverses amyloid β functional toxicity on cone cells. In some embodiments, Compound 1 reverses amyloid β functional toxicity on rod cells.

In some embodiments, Compound 2 reverses amyloid β functional toxicity. In some embodiments, Compound 2 reverses amyloid β functional toxicity in vivo. In some embodiments, Compound 2 reverses amyloid β functional toxicity on neuronal cells, on non-neuronal cells, or on neuro-sensory cells. In some embodiments, Compound 2 reverses amyloid β functional toxicity on retinal ganglion cells (RGC). In some embodiments, Compound 2 reverses amyloid β functional toxicity on retinal pigment epithelium cells (RPE). In some embodiments, Compound 2 reverses amyloid β functional toxicity on cone cells. In some embodiments, Compound 2 reverses amyloid β functional toxicity on rod cells.

In some embodiments, Compound 3 reverses amyloid β functional toxicity. In some embodiments, Compound 3 reverses amyloid β functional toxicity in vivo. In some embodiments, Compound 3 reverses amyloid β functional toxicity on neuronal cells, on non-neuronal cells, or on neuro-sensory cells. In some embodiments, Compound 3 reverses amyloid β functional toxicity on retinal ganglion cells (RGC). In some embodiments, Compound 3 reverses amyloid β functional toxicity on retinal pigment epithelium cells (RPE). In some embodiments, Compound 3 reverses amyloid β functional toxicity on cone cells. In some embodiments, Compound 3 reverses amyloid β functional toxicity on rod cells.

In some embodiments, Compound 4 reverses amyloid β functional toxicity. In some embodiments, Compound 4 reverses amyloid β functional toxicity in vivo. In some embodiments, Compound 4 reverses amyloid β functional toxicity on neuronal cells, on non-neuronal cells, or on neuro-sensory cells. In some embodiments, Compound 4 reverses amyloid β functional toxicity on retinal ganglion cells (RGC). In some embodiments, Compound 4 reverses amyloid β functional toxicity on retinal pigment epithelium cells (RPE). In some embodiments, Compound 4 reverses amyloid β functional toxicity on cone cells. In some embodiments, Compound 4 reverses amyloid β functional toxicity on rod cells.

In some embodiments, in methods disclosed herein, Compound 1, 2, 3, or 4 reverses amyloid β functional toxicity in a subject in need. A skilled artisan would appreciate that in some embodiments, reversal of amyloid β functional toxicity encompasses restoration of function.

Reversal of amyloid β functional toxicity, may in some embodiments, result in rapid restoration of impaired neuronal function. In some embodiments, neuronal function comprises response to light that affect the cells of the sensory organs (e.g., eyes) and sends signals to the spinal cord or brain. In some embodiments, neuronal function comprises receiving signals from the brain and spinal cord in order to control everything from muscle contractions to glandular output. In some embodiments, neuronal function comprises sending or receiving a signal, for example but not limited to an action potential (electric potential).

In some embodiments, restoration of an impaired function comprises restoration of a response to light. In some embodiments, restoration of an impaired function comprises restoration of the ability to send an electrical potential. In some embodiments, restoration of an impaired function comprises restoration of the ability to receive an electrical potential.

A skilled artisan would appreciate the restoration of neuronal function may be rapid, wherein the restoration of ability to send or receive an electrical potential occurs within minutes. Restoration of neuronal function being rapid would be appreciated by one skilled in the art to be rapid within the context of a disease or condition. In some embodiments, rapid restoration of neuronal function, for example the restoration of ability to send or receive an electrical potential occurs within hours. In some embodiments, rapid restoration of neuronal function, for example the restoration of ability to send or receive an electrical potential occurs within days. In some embodiments, rapid restoration of neuronal function, for example the restoration of ability to send or receive an electrical potential occurs within months. Non-invasive methods to detect restoration of neuronal function, for example in the retina of the eye are known in the art and include but are not limited to, microperimetry, measurement of low luminance visual acuity, measurement of dark adaptation, and measurement of low luminance reading speed.

In some embodiments, restoration of neuronal function is between 25-100% restoration. In some embodiments, restoration of neuronal function is between 50-100% restoration. In some embodiments, restoration of neuronal function is between 75-100% restoration. In some embodiments, restoration of neuronal function is between 50-75% restoration. In some embodiments, restoration of neuronal function comprises at least 25% restoration. In some embodiments, restoration of neuronal function comprises at least 35% restoration. In some embodiments, restoration of neuronal function comprises at least 45% restoration. In some embodiments, restoration of neuronal function comprises at least 55% restoration. In some embodiments, restoration of neuronal function comprises at least 65% restoration. In some embodiments, restoration of neuronal function comprises at least 75% restoration. In some embodiments, restoration of neuronal function comprises at least 85% restoration. In some embodiments, restoration of neuronal function comprises at least 95% restoration.

In some embodiments, restoration of neuronal function comprises about 25%-35% restoration. In some embodiments, restoration of neuronal function comprises about 35%-45% restoration. In some embodiments, restoration of neuronal function comprises about 45%-55% restoration. In some embodiments, restoration of neuronal function comprises about 55%-65% restoration. In some embodiments, restoration of neuronal function comprises about 65%-75% restoration. In some embodiments, restoration of neuronal function comprises about 75%-85% restoration. In some embodiments, restoration of neuronal function comprises about 85%-95% restoration. In some embodiments, restoration of neuronal function comprises about 90%-100% restoration.

In some embodiments, restoration of neuronal function comprises about 25% restoration. In some embodiments, restoration of neuronal function comprises about 35% restoration. In some embodiments, restoration of neuronal function comprises about 45% restoration. In some embodiments, restoration of neuronal function comprises about 55% restoration. In some embodiments, restoration of neuronal function comprises about 65% restoration. In some embodiments, restoration of neuronal function comprises about 75% restoration. In some embodiments, restoration of neuronal function comprises about 85% restoration. In some embodiments, restoration of neuronal function comprises about 95% restoration. In some embodiments, restoration of neuronal function comprises about 100% restoration.

Reversal of amyloid β functional toxicity, may in some embodiments, result in decreased cell death of neuronal, non-neuronal, and or neuro-sensory cells, or a combination thereof. In some embodiments, methods disclosed herein decrease cell death of neuronal cells. In some embodiments, methods disclosed herein decrease cell death of RGC. In some embodiments, methods disclosed herein decrease cell death of non-neuronal cells. In some embodiments, methods disclosed herein decrease cell death of RPE cells. In some embodiments, methods disclosed herein decrease cell death of astrocytes. In some embodiments, methods disclosed herein decrease cell death of neuro-sensory cells. Non-invasive methods to detect cell death, for example in the eye are known in the art and include but are not limited to, fundus autofluorescence photography and detection of apoptosing retinal cells (DARC).

In some embodiments, compounds of Formulae (I), (IA), (II), or (IIA) bind misfolded toxic Aβ₁₋₄₂ monomers. In some embodiments, binding of a compound of Formulae (I), (IA), (II), or (IIA) to Aβ₁₋₄₂ is with higher affinity than the misfolded Aβ₁₋₄₂ monomers have for each other. Binding of compounds of Formulae (I), (IA), (II), or (IIA) to misfolded toxic Aβ₁₋₄₂ monomers leads to formation of innocuous non-toxic clusters of misfolded amyloid β monomers (amorphous Aβ) that may be removed naturally from circulation or from intra- and extra-cellular spaces. Further in some embodiments, this binding to misfolded toxic Aβ₁₋₄₂ monomers does not interfere with normal the function of Amyloid β or otherwise cause toxicity.

In some embodiments, compounds of Formulae (I), (IA), (II), or (IIA) bind misfolded toxic Aβ₁₋₄₂ monomers. In some embodiments, any of compound 1-25 binds misfolded toxic Aβ₁₋₄₂ monomers. In some embodiments, Compound 1 binds misfolded toxic Aβ₁₋₄₂ monomers. In some embodiments, Compound 2 binds misfolded toxic Aβ₁₋₄₂ monomers. In some embodiments, Compound 3 binds misfolded toxic Aβ₁₋₄₂ monomers. In some embodiments, Compound 4 binds misfolded toxic Aβ₁₋₄₂ monomers.

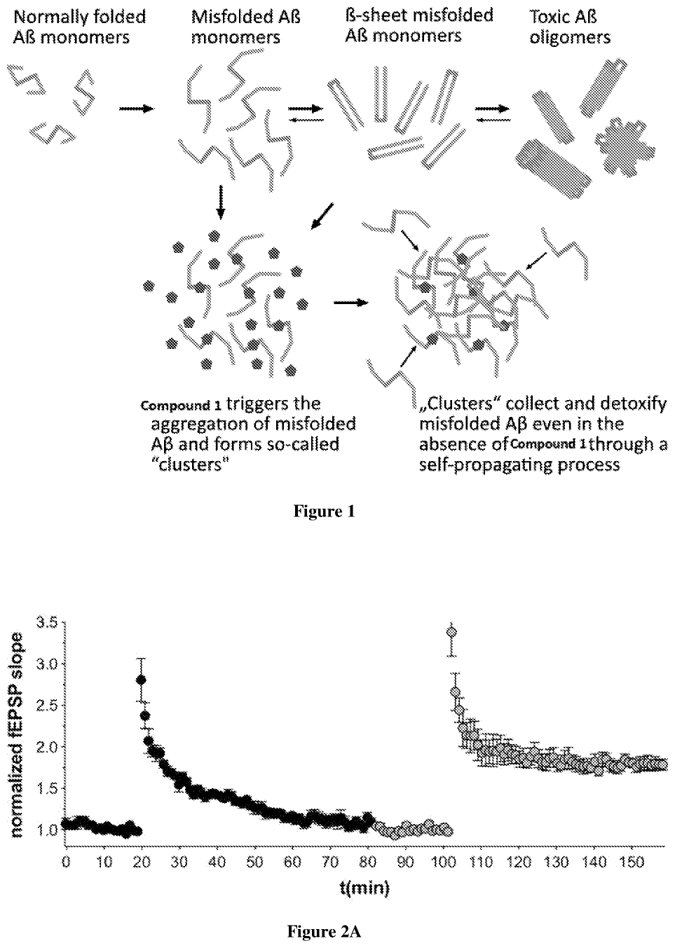

In some embodiments, compounds of Formulae (I), (IA), (II), or (IIA) form amorphous Aβ clusters in the presence of pre-existing toxic Aβ₁₋₄₂, thereby reversing the toxicity of Aβ₁₋₄₂ (FIG. 1). These amorphous Aβ clusters comprises non-toxic, non-β-sheet, amorphous Aβ clusters. As used throughout, the term “non-toxic, non-β-sheet, amorphous Aβ clusters” may be used interchangeably with “Aβ blobs”, “blobs”, “Aβ assemblies”, “assemblies”, “non-toxic Aβ aggregates”, “non-toxic aggregates”, “non-toxic Aβ clusters”, “non-toxic cluster”, “amorphous clusters”, “amorphous Aβ clusters”, “amorphous aggregates”, “amorphous Aβ aggregates” or “Aβ clusters”, or the like, having all the same meanings and qualities. In each case, one skilled in the art would appreciate that the non-toxic, non-β-sheet, amorphous Aβ clusters comprise non-toxic formations of amyloid β. In some embodiments, these clusters possess the potential for prevention of toxic Aβ oligomer formation. In some embodiments, these clusters possess the potential for the reversal of toxic Aβ oligomer formation, as seen by the reversal of functional Aβ toxicity exemplified in Example 2 below.

In some embodiments, compounds of Formulae (I), (IA), (II), or (IIA) form amorphous Aβ clusters in the presence of pre-existing toxic Aβ₁₋₄₂, thereby reversing the toxicity of Aβ₁₋₄₂ in vivo. In some embodiments, compounds of Formulae (I), (IA), (II), or (IIA) form amorphous Aβ clusters in the presence of pre-existing toxic Aβ₁₋₄₂, thereby reversing the toxicity of Aβ₁₋₄₂ on neuronal cells, in non-neuronal cells, or on neuro-sensory cells. In some embodiments, compounds of Formulae (I), (IA), (II), or (IIA) form amorphous Aβ clusters in the presence of pre-existing toxic Aβ₁₋₄₂, thereby reversing the toxicity of Aβ₁₋₄₂ on RGCs. In some embodiments, compounds of Formulae (I), (IA), (II), or (IIA) form amorphous Aβ clusters in the presence of pre-existing toxic Aβ₁₋₄₂, thereby reversing the toxic buildup of Aβ₁₋₄₂ on RPEs/Bruch's membrane.

In some embodiments, any one of compounds 1-25 forms amorphous Aβ clusters in the presence of pre-existing toxic Aβ₁₋₄₂, thereby reversing the toxicity of Aβ₁₋₄₂. In some embodiments, compounds of any one of compounds 1-25 form amorphous Aβ clusters in the presence of pre-existing toxic Aβ₁₋₄₂, thereby reversing the toxicity of Aβ₁₋₄₂ in vivo. In some embodiments, compounds of any one of compounds 1-25 form amorphous Aβ clusters in the presence of pre-existing toxic Aβ₁₋₄₂, thereby reversing the toxicity of Aβ₁₋₄₂ on neuronal cells, in non-neuronal cells, or on neuro-sensory cells. In some embodiments, compounds of any one of compounds 1-25 form amorphous Aβ clusters in the presence of pre-existing toxic Aβ₁₋₄₂, thereby reversing the toxicity of Aβ₁₋₄₂ on RGCs. In some embodiments, compounds of any one of compounds 1-25 form amorphous Aβ clusters in the presence of pre-existing toxic Aβ₁₋₄₂, thereby reversing the toxic buildup of Aβ₁₋₄₂ on RPEs/Bruch's membrane. In some embodiments, compounds of any one of compounds 1-25 form amorphous Aβ clusters in the presence of pre-existing toxic Aβ₁₋₄₂, thereby reversing the toxicity of Aβ₁₋₄₂ on cone cells. In some embodiments, compounds of any one of compounds 1-28 form amorphous Aβ clusters in the presence of pre-existing toxic Aβ₁₋₄₂, thereby reversing the toxicity of Aβ₁₋₄₂ on rod cells.

In some embodiments, Compound 1 forms amorphous Aβ clusters in the presence of pre-existing toxic Aβ₁₋₄₂, thereby reversing the toxicity of Aβ₁₋₄₂. In some embodiments, Compound 1 form amorphous Aβ clusters in the presence of pre-existing toxic Aβ₁₋₄₂, thereby reversing the toxicity of Aβ₁₋₄₂ in vivo. In some embodiments, Compound 1 form amorphous Aβ clusters in the presence of pre-existing toxic Aβ₁₋₄₂, thereby reversing the toxicity of Aβ₁₋₄₂ on neuronal cells, in non-neuronal cells, or on neuro-sensory cells. In some embodiments, Compound 1 form amorphous Aβ clusters in the presence of pre-existing toxic Aβ₁₋₄₂, thereby reversing the toxicity of Aβ₁₋₄₂ on RGCs. In some embodiments, Compound 1 form amorphous Aβ clusters in the presence of pre-existing toxic Aβ₁₋₄₂, thereby reversing the toxic buildup of Aβ₁₋₄₂ on RPEs/Bruch's membrane. In some embodiments, Compound 1 forms amorphous Aβ clusters in the presence of pre-existing toxic Aβ₁₋₄₂, thereby reversing the toxicity of Aβ₁₋₄₂ on cone cells. In some embodiments, Compound 1 forms amorphous Aβ clusters in the presence of pre-existing toxic Aβ₁₋₄₂, thereby reversing the toxicity of Aβ₁₋₄₂ on rod cells.

In some embodiments, Compound 2 forms amorphous Aβ clusters in the presence of pre-existing toxic Aβ₁₋₄₂, thereby reversing the toxicity of Aβ₁₋₄₂. In some embodiments, Compound 2 form amorphous Aβ clusters in the presence of pre-existing toxic Aβ₁₋₄₂, thereby reversing the toxicity of Aβ₁₋₄₂ in vivo. In some embodiments, Compound 2 form amorphous Aβ clusters in the presence of pre-existing toxic Aβ₁₋₄₂, thereby reversing the toxicity of Aβ₁₋₄₂ on neuronal cells, in non-neuronal cells, or on neuro-sensory cells. In some embodiments, Compound 2 form amorphous Aβ clusters in the presence of pre-existing toxic Aβ₁₋₄₂, thereby reversing the toxicity of Aβ₁₋₄₂ on RGCs. In some embodiments, Compound 2 form amorphous Aβ clusters in the presence of pre-existing toxic Aβ₁₋₄₂, thereby reversing the toxic buildup of Aβ₁₋₄₂ on RPEs/Bruch's membrane. In some embodiments, Compound 2 forms amorphous Aβ clusters in the presence of pre-existing toxic Aβ₁₋₄₂, thereby reversing the toxicity of Aβ₁₋₄₂ on cone cells. In some embodiments, Compound 2 forms amorphous Aβ clusters in the presence of pre-existing toxic Aβ₁₋₄₂, thereby reversing the toxicity of Aβ₁₋₄₂ on rod cells.

In some embodiments, Compound 3 forms amorphous Aβ clusters in the presence of pre-existing toxic Aβ₁₋₄₂, thereby reversing the toxicity of Aβ₁₋₄₂. In some embodiments, Compound 3 form amorphous Aβ clusters in the presence of pre-existing toxic Aβ₁₋₄₂, thereby reversing the toxicity of Aβ₁₋₄₂ in vivo. In some embodiments, Compound 3 form amorphous Aβ clusters in the presence of pre-existing toxic Aβ₁₋₄₂, thereby reversing the toxicity of Aβ₁₋₄₂ on neuronal cells, in non-neuronal cells, or on neuro-sensory cells. In some embodiments, Compound 3 form amorphous Aβ clusters in the presence of pre-existing toxic Aβ₁₋₄₂, thereby reversing the toxicity of Aβ₁₋₄₂ on RGCs. In some embodiments, Compound 3 form amorphous Aβ clusters in the presence of pre-existing toxic Aβ₁₋₄₂, thereby reversing the toxic buildup of Aβ₁₋₄₂ on RPEs/Bruch's membrane. In some embodiments, Compound 3 forms amorphous Aβ clusters in the presence of pre-existing toxic Aβ₁₋₄₂, thereby reversing the toxicity of Aβ₁₋₄₂ on cone cells. In some embodiments, Compound 3 forms amorphous Aβ clusters in the presence of pre-existing toxic Aβ₁₋₄₂, thereby reversing the toxicity of Aβ₁₋₄₂ on rod cells.

In some embodiments, Compound 4 forms amorphous Aβ clusters in the presence of pre-existing toxic Aβ₁₋₄₂, thereby reversing the toxicity of Aβ₁₋₄₂. In some embodiments, Compound 4 form amorphous Aβ clusters in the presence of pre-existing toxic Aβ₁₋₄₂, thereby reversing the toxicity of Aβ₁₋₄₂ in vivo. In some embodiments, Compound 4 form amorphous Aβ clusters in the presence of pre-existing toxic Aβ₁₋₄₂, thereby reversing the toxicity of Aβ₁₋₄₂ on neuronal cells, in non-neuronal cells, or on neuro-sensory cells. In some embodiments, Compound 4 form amorphous Aβ clusters in the presence of pre-existing toxic Aβ₁₋₄₂, thereby reversing the toxicity of Aβ₁₋₄₂ on RGCs. In some embodiments, Compound 4 form amorphous Aβ clusters in the presence of pre-existing toxic Aβ₁₋₄₂, thereby reversing the buildup of Aβ₁₋₄₂ on RPEs/Bruch's membrane. In some embodiments, Compound 4 forms amorphous Aβ clusters in the presence of pre-existing toxic Aβ₁₋₄₂, thereby reversing the toxicity of Aβ₁₋₄₂ on cone cells. In some embodiments, Compound 4 forms amorphous Aβ clusters in the presence of pre-existing toxic Aβ₁₋₄₂, thereby reversing the toxicity of Aβ₁₋₄₂ on rod cells.

In some embodiments, compounds of Formulae (I), (IA), (II), or (IIA) remove toxic amyloid β deposits from cell surfaces. In some embodiments, compounds of Formulae (I), (IA), (II), or (IIA) reduces amyloid β deposits from cell surfaces. In some embodiments, use of compounds of Formulae (I), (IA), (II), or (IIA) leads to formation of amorphous aggregates of amyloid beta along cell surfaces. In some embodiments, use of compounds of Formulae (I), (IA), (II), or (IIA) leads to formation of amorphous aggregates of amyloid beta along in neuronal cell surfaces. In some embodiments, use of compounds of Formulae (I), (IA), (II), or (IIA) leads to formation of amorphous aggregates of amyloid beta along non-neuronal cell surfaces. In some embodiments, use of compounds of Formulae (I), (IA), (II), or (IIA) leads to formation of amorphous aggregates of amyloid beta along neuro-sensory cell surfaces. In some embodiments, use of compounds of Formulae (I), (IA), (II), or (IIA) leads to formation of amorphous aggregates of amyloid beta along retinal ganglion cells (RGC). In some embodiments, use of compounds of Formulae (I), (IA), (II), or (IIA) leads to formation of amorphous aggregates of amyloid beta along retinal pigment epithelium cells (RPE)/Bruch's membrane. In some embodiments, use of compounds of Formulae (I), (IA), (II), or (IIA) leads to formation of amorphous aggregates of amyloid beta along cone cells. In some embodiments, use of compounds of Formulae (I), (IA), (II), or (IIA) leads to formation of amorphous aggregates of amyloid beta along rod cells.

In some embodiments, any one of compounds 1-25 removes toxic amyloid β deposits from cell surfaces. In some embodiments, any one of compounds 1-25 reduces amyloid β deposits from cell surfaces. In some embodiments, use of any one of compounds 1-25 leads to formation of amorphous aggregates of amyloid beta along cell surfaces. In some embodiments, use of any one of compounds 1-25 leads to formation of amorphous aggregates of amyloid beta along in neuronal cell surfaces. In some embodiments, use of any one of compounds 1-25 leads to formation of amorphous aggregates of amyloid beta along non-neuronal cell surfaces. In some embodiments, use of any one of compounds 1-25 leads to formation of amorphous aggregates of amyloid beta along neuro-sensory cell surfaces. In some embodiments, use of any one of compounds 1-25 leads to formation of formation amorphous aggregates of amyloid beta along retinal ganglion cells (RGC). In some embodiments, use of any one of compounds 1-25 leads to formation of formation amorphous aggregates of amyloid beta along retinal pigment epithelium cells (RPE)/Bruch's membrane. In some embodiments, use of any one of compounds 1-25 leads to formation of formation amorphous aggregates of amyloid beta along cone cells. In some embodiments, use of any one of compounds 1-25 leads to formation of formation amorphous aggregates of amyloid beta along rod cells.

In some embodiments, Compound 1 removes toxic amyloid β deposits from cell surfaces. In some embodiments, Compound 1 reduces amyloid β deposits from cell surfaces. In some embodiments, use of Compound 1 leads to formation of amorphous aggregates of amyloid beta along cell surfaces. In some embodiments, use of Compound 1 leads to formation of amorphous aggregates of amyloid beta along in neuronal cell surfaces. In some embodiments, use of Compound 1 leads to formation of amorphous aggregates of amyloid beta along non-neuronal cell surfaces. In some embodiments, use of Compound 1 leads to formation of amorphous aggregates of amyloid beta along neuro-sensory cell surfaces. In some embodiments, use of Compound 1 leads to formation of amorphous aggregates of amyloid beta along retinal ganglion cells (RGC). In some embodiments, use of Compound 1 leads to formation of formation amorphous aggregates of amyloid beta along retinal pigment epithelium cells (RPE)/Bruch's membrane. In some embodiments, use of Compound 1 leads to formation of formation amorphous aggregates of amyloid beta along cone cells. In some embodiments, use of Compound 1 leads to formation of amorphous aggregates of amyloid beta along rod cells.

In some embodiments, Compound 2 removes toxic amyloid β deposits from cell surfaces. In some embodiments, Compound 2 reduces amyloid β deposits from cell surfaces. In some embodiments, use of Compound 2 leads to formation of amorphous aggregates of amyloid beta along cell surfaces. In some embodiments, use of Compound 2 leads to formation of amorphous aggregates of amyloid beta along in neuronal cell surfaces. In some embodiments, use of Compound 2 leads to formation of amorphous aggregates of amyloid beta along non-neuronal cell surfaces. In some embodiments, use of Compound 2 leads to formation of amorphous aggregates of amyloid beta along neuro-sensory cell surfaces. In some embodiments, use of Compound 2 leads to formation of amorphous aggregates of amyloid beta along retinal ganglion cells (RGC). In some embodiments, use of Compound 2 leads to formation of amorphous aggregates of amyloid beta along retinal pigment epithelium cells (RPE)/Bruch's membrane. In some embodiments, use of Compound 2 leads to formation of formation amorphous aggregates of amyloid beta along cone cells. In some embodiments, use of Compound 2 leads to formation of amorphous aggregates of amyloid beta along rod cells.

In some embodiments, Compound 3 removes toxic amyloid β deposits from cell surfaces. In some embodiments, Compound 3 reduces amyloid β deposits from cell surfaces. In some embodiments, use of Compound 3 leads to formation of amorphous aggregates of amyloid beta along cell surfaces. In some embodiments, use of Compound 3 leads to formation of amorphous aggregates of amyloid beta along in neuronal cell surfaces. In some embodiments, use of Compound 3 leads to formation of amorphous aggregates of amyloid beta along non-neuronal cell surfaces. In some embodiments, use of Compound 3 leads to formation of amorphous aggregates of amyloid beta along neuro-sensory cell surfaces. In some embodiments, use of Compound 3 leads to formation of amorphous aggregates of amyloid beta along retinal ganglion cells (RGC). In some embodiments, use of Compound 3 leads to formation of amorphous aggregates of amyloid beta along retinal pigment epithelium cells (RPE)/Bruch's membrane. In some embodiments, use of Compound 3 leads to formation of amorphous aggregates of amyloid beta along cone cells. In some embodiments, use of Compound 3 leads to formation of amorphous aggregates of amyloid beta along rod cells.

In some embodiments, Compound 4 removes toxic amyloid β deposits from cell surfaces. In some embodiments, Compound 4 reduces amyloid β deposits from cell surfaces. In some embodiments, use of Compound 4 leads to formation of amorphous aggregates of amyloid beta along cell surfaces. In some embodiments, use of Compound 4 leads to formation of amorphous aggregates of amyloid beta along in neuronal cell surfaces. In some embodiments, use of Compound 4 leads to formation of amorphous aggregates of amyloid beta along non-neuronal cell surfaces. In some embodiments, use of Compound 4 leads to formation of amorphous aggregates of amyloid beta along neuro-sensory cell surfaces. In some embodiments, use of Compound 4 leads to formation of amorphous aggregates of amyloid beta along retinal ganglion cells (RGC). In some embodiments, use of Compound 4 leads to formation of amorphous aggregates of amyloid beta along retinal pigment epithelium cells (RPE)/Bruch's membrane. In some embodiments, use of Compound 4 leads to formation of amorphous aggregates of amyloid beta along cone cells. In some embodiments, use of Compound 4 leads to formation of amorphous aggregates of amyloid beta along rod cells.

In some embodiments, compounds of Formulae (I), (IA), (II), or (IIA) reverse the inhibition of Long-Term Potentiation (LTP) caused by pre-existing toxic Aβ₁₋₄₂ aggregates, thereby reversing the toxicity of Aβ₁₋₄₂. In some embodiments, compounds of Formulae (I), (IA), (II), or (IIA) reverse the inhibition of Long-Term Potentiation (LTP) caused by pre-existing toxic Aβ₁₋₄₂ aggregates in vivo. In some embodiments, compounds of Formulae (I), (IA), (II), or (IIA) reverse the inhibition of Long-Term Potentiation (LTP) caused by pre-existing toxic Aβ₁₋₄₂ aggregates in neuro-sensory cells.

In some embodiments, any of compounds 1-25 reverse the inhibition of Long-Term Potentiation (LTP) caused by pre-existing toxic Aβ₁₋₄₂ aggregates. thereby reversing the toxicity of Aβ₁₋₄₂. In some embodiments, any of compounds 1-25 reverse the inhibition of Long-Term Potential (LTP) caused by pre-existing toxic Aβ₁₋₄₂ aggregates in vivo. In some embodiments, any of compounds 1-25 reverse the inhibition of Long-Term Potentiation (LTP) caused by pre-existing toxic Aβ₁₋₄₂ aggregates in neuro-sensory cells. In some embodiments, the neuro-sensory cells comprise RGCs, RPE cells, cone cells, and rod cells

In some embodiments, Compound 1 reverses the inhibition of Long-Term Potential (LTP) caused by pre-existing toxic Aβ₁₋₄₂ aggregates. Thereby reversing the toxicity of Aβ₁₋₄₂ oligomers. In some embodiments, Compound 1 reverses the inhibition of Long-Term Potential (LTP) caused by pre-existing toxic Aβ₁₋₄₂ aggregates in vivo. In some embodiments, Compound 1 reverses the inhibition of Long-Term Potential (LTP) caused by pre-existing toxic Aβ₁₋₄₂ aggregates in neuronal, non-neuronal, and neuro-sensory cells. In some embodiments, the neuronal, non-neuronal, and neuro-sensory cells comprise RGCs.

In some embodiments, Compound 2 reverses the inhibition of Long-Term Potential (LTP) caused by pre-existing toxic Aβ₁₋₄₂ aggregates. Thereby reversing the toxicity of Aβ₁₋₄₂ oligomers. In some embodiments, Compound 2 reverses the inhibition of Long-Term Potential (LTP) caused by pre-existing toxic Aβ₁₋₄₂ aggregates in vivo. In some embodiments, Compound 2 reverses the inhibition of Long-Term Potential (LTP) caused by pre-existing toxic Aβ₁₋₄₂ aggregates in neuronal, non-neuronal, and neuro-sensory cells. In some embodiments, the neuronal, non-neuronal, and neuro-sensory cells comprise RGCs.

In some embodiments, Compound 3 reverses the inhibition of Long-Term Potential (LTP) caused by pre-existing toxic Aβ₁₋₄₂ aggregates. thereby reversing the toxicity of Aβ₁₋₄₂. In some embodiments, Compound 3 reverses the inhibition of Long-Term Potential (LTP) caused by pre-existing toxic Aβ₁₋₄₂ aggregates in vivo. In some embodiments, Compound 3 reverses the inhibition of Long-Term Potential (LTP) caused by pre-existing toxic Aβ₁₋₄₂ aggregates in neuronal, non-neuronal, and neuro-sensory cells. In some embodiments, the neuronal, non-neuronal, and neuro-sensory cells comprise RGCs.

In some embodiments, Compound 4 reverses the inhibition of Long-Term Potential (LTP) caused by pre-existing toxic Aβ₁₋₄₂ aggregates. Thereby reversing the toxicity of Aβ₁₋₄₂. In some embodiments, Compound 4 reverses the inhibition of Long-Term Potential (LTP) caused by pre-existing toxic Aβ₁₋₄₂ aggregates in vivo. In some embodiments, Compound 4 reverses the inhibition of Long-Term Potential (LTP) caused by pre-existing toxic Aβ₁₋₄₂ aggregates in neuronal, non-neuronal, and neuro-sensory cells. In some embodiments, the neuronal, non-neuronal, and neuro-sensory cells comprise RGCs.

In some embodiments, in methods described herein the subject is suffering from an amyloid β-associated disease or condition. One skilled in the art would appreciate that an amyloid β-associated disease or condition encompasses a group of diseases in which abnormal proteins, known as amyloid fibrils, builds up in tissue. For example, but not limited to, in some embodiments, an amyloid β-associated disease or condition comprises an optical or neurological disease or condition.

In some embodiments, an amyloid β ophthalmic disease or condition comprises primary angle-closure glaucoma, secondary open-angle glaucoma, wide-angle glaucoma, steroid-induced glaucoma, traumatic glaucoma, pigmentary dispersion syndrome, pseudo-exfoliation syndrome, secondary angle-closure glaucoma, neovascular glaucoma, early and intermediate dry (non-exudative) age-related macular degeneration, macular degeneration with geographic atrophy, exudative (“wet”) macular degeneration, or diabetic retinopathy, or a combination thereof. In some embodiments, methods disclosed herein reversing amyloid β functional toxicity improve relatively rapidly visual acuity, low luminescence vision, contrast sensitivity, cone contrast sensitivity, color vision, focal and general retinal light sensitivity in photopic mesopic (light adaptation) and scotopic (dark adaptation) conditions, and indirectly also postural stability, gait balance and mobility, in said subject.

When methods of use described herein are implemented in a subject suffering from all types of glaucoma, reversal of amyloid β functional toxicity of retinal eye cells, for example RGC or RPE, may be measured using OCT, visual field exams, microperimetry, measurement of low luminance visual acuity, measurement of dark adaptation, and measurement of low luminance reading speed.

In some embodiments, an amyloid β neurological disease or condition comprises type II diabetes mellitus, Alzheimer's disease (AD), early onset Alzheimer's disease, late onset Alzheimer's disease, pre-symptomatic Alzheimer's disease, SAA amyloidosis, hereditary Icelandic syndrome, multiple myeloma, medullary carcinoma, aortic medical amyloid, Insulin injection amyloidosis, prion-systemic amyloidosis, chronic inflammation amyloidosis, senile systemic amyloidosis, pituitary gland amyloidosis, hereditary renal amyloidosis, familial British dementia, Finnish hereditary amyloidosis, familial non-neuropathic amyloidosis, and disorders and prion diseases, or a combination thereof.

In some embodiments, an amyloid β neurological disease or condition comprises diabetes mellitus. In some embodiments, an amyloid β neurological disease or condition comprises type II diabetes mellitus.

When the neurological disease comprises Alzheimer's disease (AD), early onset Alzheimer's disease, late onset Alzheimer's disease, or pre-symptomatic Alzheimer's disease, in some embodiments, methods disclosed herein provide improvement of cognitive deficiencies, improvement memory loss, reduction of abnormal behavior, reduction of hallucinations, reduction of loss of spatial orientation, reduction of apraxia, reduction of aggression, improvement in the ability to perform activities of daily living, or other symptoms of dementia, or any combination thereof, in said subject.

Due to their high degree of biological activity and their low local and systemic toxicity, together presenting a favorable therapeutic index, the compounds of Formula (I), (IA), (II) or (IIA) or any one of compounds 1-25 may be administered to a subject, e.g., a living mammal (including a human) body, for the treatment, alleviation, amelioration, palliation, reversal, or elimination of a symptom, an indication, or condition, which is susceptible thereto, or representatively of an indication or condition set forth elsewhere in this application, preferably concurrently, simultaneously, or together with one or more pharmaceutically acceptable excipients, especially in the form of a pharmaceutical composition thereof, whether by oral, rectal, parental, or topical route, in an effective amount. In some embodiments, a compound disclosed herein is administered by oral, topical, nasal administration. In some embodiments, a compound disclosed herein is administered by intravenous, subcutaneous, implanted slow-release depots, direct injection using an in-dwelling catheter, intrathecal, or intraocular injection.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Suitable dosage ranges are 1 to 1000 milligrams daily, preferably 5 to 500 milligrams daily, and especially 10 to 500 milligrams daily, depending as usual upon the exact mode of administration, form in which administered, the indication toward which the administration is directed, the subject involved and the body weight of the subject involved, and the preference and experience of the physician or veterinarian in charge. In one embodiment, the term “therapeutically effective” applied to dose or amount refers to that quantity of a compound or pharmaceutical composition that is sufficient to result in a desired activity upon administration to a living animal body in need thereof.

In some embodiments, the compounds of formula (I), (IA), (II) or (IIA), or any of compounds 1-25 for use in the methods described herein may be administered orally, nasally, topically, parenterally, or mucosally (e.g., buccally, by inhalation, or rectally) in dosage unit formulations containing conventional non-toxic pharmaceutically acceptable excipients. In some embodiments, the compounds of formula (I), (IA), (II) or (IIA), or any of compounds 1-25 for use in the methods described herein may be administered by intravenous, subcutaneous, implanted slow-release depots, direct injection using an in-dwelling catheter, intrathecal, or intraocular injection, in dosage unit formulations containing conventional non-toxic pharmaceutically acceptable excipients.

In some embodiments, administration is in the form of multiple doses administered over a period of time, wherein said time period comprises days, weeks, or months, or at most 1 year. In some embodiments, administration is in the form of multiple doses administered over 1-7 days. In some embodiments, administration is in the form of multiple doses administered over 1-4 weeks. In some embodiments, administration is in the form of multiple doses administered over 1-12 months. In some embodiments, administration is in the form of multiple doses administered over at most 1 year or several over years. In some embodiments, administration is in the form of multiple doses administered over the life-time of the subject. In some embodiments, administration is in the form of multiple doses administered as long as the amyloid β functional toxicity persists, wherein administration is required to reverse the persistence of the toxicity. In some embodiments, administration is in the form of multiple doses administered as long as the amyloid β functional toxicity persists, wherein administration is required to reduce the toxicity.

In some embodiments, a method of use disclosed herein comprises administering a compound disclosed herein in a pattern of dosage within a time period. In some embodiments, the administration may be at regular intervals, or at irregular intervals, or a combination thereof. In some embodiments, the administration may be at regular intervals. In some embodiments, the administration may be at irregular intervals. Some embodiments of interval intermittent treatment are described in detail in publication WO 13/18960, which is incorporated herein in its entirety.

As used herein, the phrase “Intermittent interval administration” encompasses specific embodiments of interval administration wherein the second dose equals a percentage (%) of the first dose. The second period will often be a longer time period than the first period. For example, the first period may be one day, and the second period may be one or more weeks, or one or more months; or the first period will be one week, and the second period will be two or more weeks, or one or more months. Often, the second period will be less than or equal to a year. In some embodiments, the interval or a portion thereof, repeat themselves.

As used herein, the phrase “Continuous administration” or “non-interval” administration encompass regular administration of doses at equal time periods.

### EXAMPLES

### Example 1: Examination of Pharmacological Properties of Compounds of Formula I in the Presence of Amyloid β₁₋₄₂

Objective:

To compare pharmacological properties of four compounds of Formula IA in the presence of Amyloid β₁₋₄₂. Specifically, the ability of Compounds 1, 2, 3, and or 4, (1) to bind Aβ₁₋₄₂, (2) to form amorphous aggregates with Aβ₁₋₄₂ (detoxify Aβ₁₋₄₂), (3) to reverse LTP inhibition cause by the presence of Aβ₁₋₄₂ in vitro and in vivo, (4) to cause depolarization of resting membrane potential, were compared.

Methods:

Methods presented here have been previously described detail, at least in: Parsons, C. G., et al. (2015), MRZ-99030—A novel modulator of Abeta aggregation: I—Mechanism of action (MoA) underlying the potential neuroprotective treatment of Alzheimer's disease, glaucoma and age-related macular degeneration (AMD). Neuropharmacology 92: 158-169. Brief descriptions are provided below. MRZ-99030 is a former code for Compound 1

Surface Plasmon Resonance

Surface plasmon resonance (SPR) experiments allow the investigation of binding of compounds to lower concentrations of Amyloid β₁₋₄₂ (Aβ₁₋₄₂) and offer the possibility to directly asses the affinity of such binding.

Atomic Force Microscopy (AFM)

AFM is one method of measuring the effect of the different compounds on the rate of loss of toxic oligomeric Aβ₁₋₄₂ species and the promotion of the formation of large, amorphous, nontoxic aggregates from Aβ₁₋₄₂.

Dynamic Light Scattering (DLS)

DLS provides another measure of the effect of the different compounds to promote the formation of large globular, non-toxic aggregates from Aβ₁₋₄₂.

Long-Term Potentiation (LTP) In Vitro and In Vivo

Details of methods to measure LTP may be found at least in: Rammes, G., Gravius, A., Ruitenberg, M., Wegener, N., Chambon, C., Sroka-Saidi, K., Jeggo, R., Staniaszek, L., Spanswick, D., O'Hare, E., Palmer, P., Kim, E. M., Bywalez, W., Egger, V. and Parsons, C. G. (2015). MRZ-99030—A novel modulator of Abeta aggregation: II—Reversal of Abeta oligomer-induced deficits in long-term potentiation (LTP) and cognitive performance in rats and mice. Neuropharmacology 92: 170-182. LTP provides a measure of synaptic activity between two neurons. MRZ-99030 is a former code for Compound 1

Results:

Table 1 below presents a comparative summary of pharmacological properties of compounds of Formula IA, that impact the effectiveness of these compounds to successfully reverse and or improve symptoms of an amyloid β disease or condition. Symptomatic improvement in an established chronic disease, such as are amyloid β diseases or conditions, may be viewed as a reversal of an existing pathology or palliative treatment of symptoms.

TABLE 1
Summary of Pharmacological Properties of Compounds 1, 2, 3, and 4.

| | Compound 1 | Compound 2 | Compound 3 | Compound 4 |
|---|---|---|---|---|
| SPR/Aβ | 29.6 ± 1.7 nm | 12.2 ± 8.5 nM | 1.4 ± 1.0 nM | 2.8 ± 1.5 nM |
| AFM/Aβ | bigger aggregates | bigger aggregates | smaller aggregates | bigger aggregates |
| DLS/Aβ | bigger aggregates | bigger aggregates | smaller aggregates | smaller aggregates |
| Patch-Clamp/Aβ | partial prevention | almost full prevention | almost full prevention | partial prevention |
| In vitro LTP/Aβ | 100 nm: full reversal | full reversal | tbd | 500 nM: no effect |
| In vivo LTP/Aβ | 50 mg/kg reversal | 0.4 mg/kg reversal | tbd | 2 mg/kg partial effect |
| | | | | 10 mg/kg deficit |

Underline = equivalent or better compared to Compound 1; double underline = different compared to Compound 1.

Summary:

The preclinical data presented in Table 1, suggests that compounds 2, 3, and 4 might have different modes of operation compared to Compound 1. In addition, the activities measure indicate that Compound 2 seems to be superior to Compound 1.

### Example 2: Reversal of Amyloid β₁₋₄₂ Functional Toxicity in the Hippocampus

Objective:

To examine the effect of compounds of Formula IA on amyloid β₁₋₄₂ (Aβ₁₋₄₂) functional toxicity in the brain, specifically the hippocampus, but also relevant for other brain areas involved in synaptic plasticity and or learning

Methods:

Brain slice preparation for field Excitatory Post Synaptic Potentials (fEPSPs) and Excitatory Post Synaptic Currents (EPSCs) recordings

The experimental protocols were approved by the ethical committee on animal care and use of the government of Bavaria, Germany. Sagittal hippocampal slices (350 mM thick) were obtained from adult (approx. 2 month) C57Bl/6 mice that were anaesthetized with isoflurane before decapitation. The head was instantly placed in ice cold Ringer solution—composition (125 mM NaCl, 2.5 mM KCl, 25 mM NaHCO₃, 2 mM CaCl₂, 1 mM MgCl₂, 25 mM D-glucose, and 1.25 mM NaH₂PO₄, bubbled with a 95% O₂/5% CO₂ mixture, and had a final pH of 7.3) saturated with carbogen gas (95% O₂, 5% CO₂; later only referred to as carbogen). Tissue was kept in this Ringer, and then used for all further procedures. The brain was removed within 1 min after decapitation, the cerebellum was cut off and the remaining brain was separated into its two hemispheres with a razor blade.

Transversal slices (350 m thick) were prepared using a microtome (HM 650 V; Microm International, Walldorf, Germany). Slices were allowed to recover at 34° C. for 45 min in standard artificial cerebrospinal fluid (aCSF) before they were transferred to the recording chamber. A platinum ring with nylon filaments was used to fix the slices on the bottom of the recording chamber, which was continuously perfused (8 mL/min) with aCSF.

Recording of fEPSPs

Extracellular recordings of fEPSP were made in the CA1 Stratum radiatum of the hippocampus using borosilicate glass micropipettes (Hugo Sachs Elektronik-Harvard Apparatus, March-Hugstetten, Germany) resulting in an open tip resistance of 1-2 MΩ, filled with aCSF. fEPSP were evoked by alternately delivering a test stimulus (50 μs, 5-20 V) via one of two bipolar tungsten electrodes (Hugo Sachs Elektronik-Harvard Apparatus, insulated to the tip; 50 μm tip diameter), placed at either side of the recording pipette, thus stimulating non-overlapping populations of the Schaffer collateral-associational commissural pathway. Stimulus frequency was 0.033 Hz per electrode.

For baseline recordings, stimulation intensity was adjusted to values evoking a response of approximately 25-30% of the maximum response. Both stimulating electrodes were used to utilize the input specificity of long-term potential (LTP) and thereby allow the measurement of an internal control within the same slice. Aβ₁₋₄₂ 50 nM was applied via the bath solution for 90 min before attempting to induce LTP following high-frequency stimulation (HFS) delivered via the first electrode.

After recording LTP for 60 mins, the bath solution was exchanged for that following serial dilution—see protocol below. This solution still contained Aβ1-42 50 nM but only 0.1 nM of Compound 1 or Compound 2. Slices were incubated for a further 90 mins before attempting to induced LTP in the second input which was then recorded for an additional 60 mins.

Control experiments confirmed that the extent of LTP did not depend on the time that slices were in the chamber, at least not for the maximal duration used in the present studies of up to 5 h. The recordings were amplified, filtered (3 kHz), and digitized (9 kHz) using a laboratory interface board (ITC-16, Instrutech Corp., NY, USA) and the “LTP program”—software (Anderson and Collingridge (2001) The LTP Program: a data acquisition program for on-line analysis of long-term potentiation and other synaptic events. Journal of Neuroscience Methods 108, 71-83.), available from http://www/ltp-program.com. Stimuli were applied in an alternating manner to each input. Two signals of the respective input were averaged to one for analysis making 1 every minute. Data were re-analyzed offline with the analysis program Igor Pro v6.1 (Wavemetrics, Lake Oswego, Oreg., USA) software. Measurements of the slope of the fEPSP were taken between 20% and 80% of the peak amplitude. Slopes of fEPSPs were normalized with respect to the 30-min control period before tetanic stimulation.

Amyloid β₁₋₄₂ (Aβ₁₋₄₂) Preparation

Aβ₁₋₄₂ (order number H-1368; Bachem, CH-Bubendorf) was suspended in 100% hexafluoroisopropanol (HFIP) (Sigma Aldrich), aliquoted to 50 μg portions and then HFIP was removed by using a Speedvac for approximately 30 min, and when completely dry, the peptides were stored at −20° C. The Aβ₁₋₄₂ was dissolved in dry DMSO (Sigma Aldrich) to a concentration of 100 μM with the aid of an ultrasonic water bath. This solution was further diluted using Ringer solution.

To test the prion-like seeding hypothesis and reversal of existing Aβ₁₋₄₂ induced deficits in LTP, a serial dilution of Compound 1 (1 μM) or Compound 2 (1 μM) was used starting with a 20:1 stoichiometric excess to Aβ₁₋₄₂ 50 nM. After incubating the Aβ1-42/Compound 1 or Compound 2 compound mixture for 20 minutes, the mixture was transferred to a freshly prepared solution with Aβ₁₋₄₂. This dilution step was repeated 5 times finally resulting in a 500:1 stoichiometric excess of Aβ1-42 over Compound 1 or of Compound 2. The final solution (containing only 0.1 nM Compound 1 or Compound 2, but still contained 50 nM of Aβ₁₋₄₂) was then tested for its ability to reverse deficits in long-term potentiation (LTP) in hippocampal slices. FIG. 6 presents a schematic of one embodiment of serial dilution steps.

All experiments were carried out at room temperature.

Results:

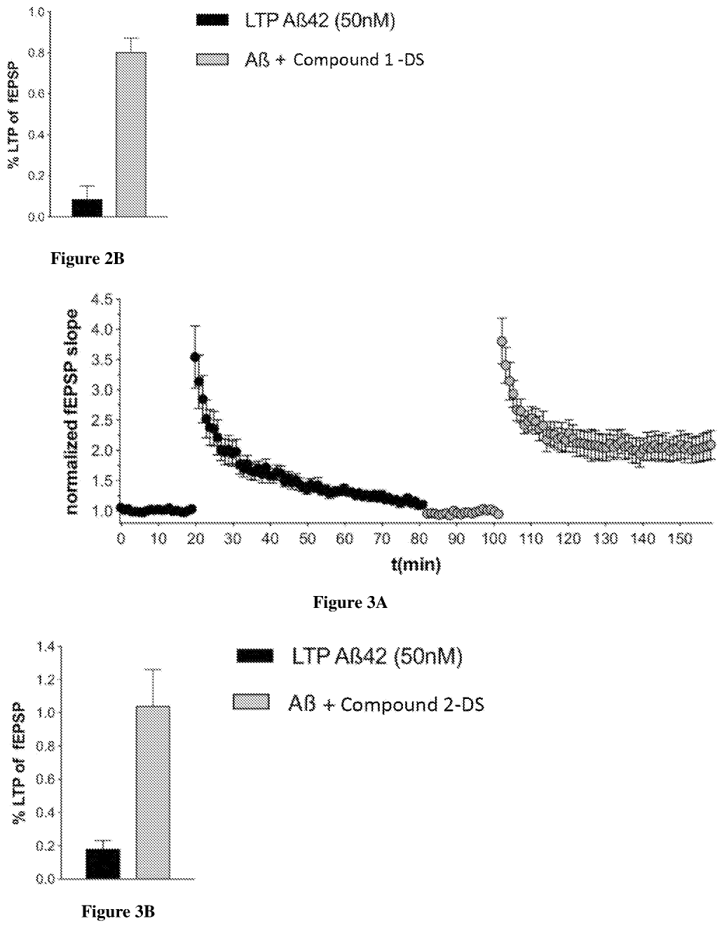

Prior incubation of hippocampal slices with Aβ₁₋₄₂ 50 nM aggregated under serial dilution conditions without Compound 1 or Compound 2 caused a strong inhibition of LTP (FIGS. 2A and 3A black circles). Surprisingly, this inhibition of LTP was reversed in the same hippocampal slices, upon addition of Aβ₁₋₄₂ 50 nM aggregated following “seeding” with Compound 1 or Compound 2 by serial dilution conditions (starting concentration of Compound 1 or Compound 2=1 μM, final concentration of Compound 1 or Compound 2=0.1 nM). Measurement of the percent differential between the LTP recordings is presented in FIGS. 2B and 3B, wherein the percent (%) LTP from the last ten minutes of the recording of either Aβ₁₋₄₂ alone or followed by Aβ₁₋₄₂ with Compound 1 or Compound 2 are compared. Significant detoxification (reversal of LTP activity) was observed in the presence of Compound of Formula X or Formula Y.

These results demonstrate reversal of the toxic effect of Aβ₁₋₄₂ on fEPSP's and corresponding neurorestoration resulting from administration of Compound 1 or Compound 2. Thus, Compound 1 and Compound 2 have the ability to reverse an existing deficit in neuroplasticity induced by Aβ. The novel aspect of these data is true reversal rather than simple prevention of these Aβ-induced deficits. In some embodiments, Compound 1 or Compound 2 restore a neurological deficit induced by Aβ. Further, LTP is a functional, electrophysiological model for the synaptic plasticity that underlies memory formation and learning. The reversal of toxic effect observed herein, is an indication of possible reversal of memory loss or improvement in learning that could be achieved with the use of Compounds 1 or 2.

Conclusion:

The reversal of the toxic effect was surprising and unexpected. These compounds were designed to bind to misfolded Aβ monomers and prevent them taking on a β-sheet structure, which normally promotes aggregation. Reversal of ongoing Aβ oligomer toxicity indicates that these compounds can additionally reverse toxicity after these oligomers have been formed. In other words, Compounds of Formula I, e.g., Compounds 1 and 2 are not classical beta-sheet breakers. Moreover, the extent of the reversal of response was unexpectedly large, e.g., a return to control levels.

### Example 3: Reduction of Toxic Aβ₁₋₄₂ by Compound of Formula IA in the Retina in a Glaucoma Mouse Model

Objective:

To examine the effect of Compound of Formula IA, for example compounds 1, 2, 3, or 4, on amyloid β₁₋₄₂ (Aβ₁₋₄₂) deposits accumulated in glaucoma.

Methods:

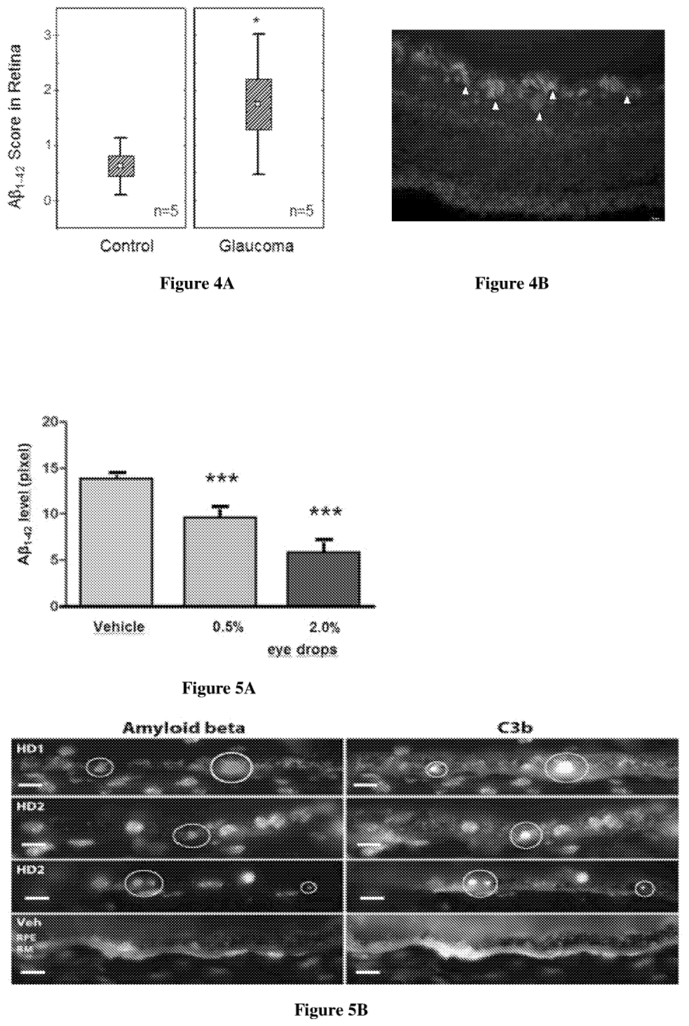

An in vivo rat model of glaucoma, the Morrison model of glaucoma, will be used to examine the reversal of the ongoing pathological process of accumulation of Aβ₁₋₄₂ along the retina and in the area of the optic nerve fiber layer, as these rats would have already had pathological changes prior to the start of treatment. FIGS. 4A and 4B show representative images of increase amyloid β in the retina of human patients versus controls (FIG. 4A) or the localization thereof by immunostaining (FIG. 4B). A similar pattern of distribution would be expected to be observed in the Morrison rat model retina.

Compounds of Formula IA, for example Compounds 1, 2, 3, or 4, will be administered to Morrison model rats, for example in the form of eye-drops and or intraocular injections. Starting concentrations of Compounds 1, 2, 3, or 4 in eye-drops would be 0.5% and 2.0%, with control eye-drops being vehicle alone.

Results:

The expected results will show a reverse of the pathology present along the retina and optic nerve fiber layer of the glaucomic eye of the Morrison model rats compared with controls.

### Example 4: Reduction of Toxic Aβ₁₋₄₂ and Complement Component C3b by Compound 1 in the Retina in an Age-Related Macular Degeneration (AMD) Mouse Model

Objective:

To examine the effect of Compound 1 on amyloid β₁₋₄₂ (Aβ₁₋₄₂) deposits accumulated in age-related Macular Degeneration (AMD).

Methods:

An in vivo mouse model of age-related Macular Degeneration, C57BL/6 (C57) mice, were used to examine the reversal of the ongoing pathological process of accumulation of Aβ₁₋₄₂ along the retina (Retinal Pigment Epithelial (RPE) cell layer/Bruch's membrane) and in the area of the optic nerve fiber layer.

Retinal Expression of Aβ₁₋₄₂ (photoreceptor layer) was analyzed in 5-6 months old AMD mice, treated three times a day for three months. Treatment method: administration of eye-drops including vehicle alone, 0.5% compound 1, or 2.0% compound 1. Reduction of toxic Aβ₁₋₄₂ deposits and complement component C3b in the retina was analyzed in 24-month-old C57BL/6 (C57) mice with heavy deposition of amyloid beta along the RPE/Bruch's membrane. Mice were treated with 0.5% Compound 1, or 2.0% Compound 1 three times a day for 1 month. Immunostaining, the enucleated eyes (n=10 from each group) were fixed in 4% paraformaldehyde in phosphate buffered saline (PBS), pH 7.4, for 1 h and were cryopreserved in 30% sucrose in PBS and embedded in OCT compound (Agar Scientific Ltd). Antibodies used were Mouse monoclonal antibody to amyloid beta (Aβ) 4G8 conjugated with Alexa Fluor 568, Goat polyclonal antibody to complement C₃, Mouse monoclonal antibody to amyloid beta (Aβ) 12F4 conjugated with Alexa Fluor 568

Results:

The mice used in both studies to analyze retinal expression and localization of Aβ₁₋₄₂, already had pathological changes to their retina prior to the start of treatment. FIG. 5A Vehicle only shows a significantly higher measure of Aβ₁₋₄₂ at the starting point compare to after administration of eye-drops comprising Compound 1. FIG. 5B bottom micrographs on each side show heavy depositions of Aβ (red fluorescence) along the RPE/Bruch's membrane. Administration of eye-drops comprising Compound 1 reduced the total amount of toxic Aβ₁₋₄₂ expression along the Bruch's membrane (BM). Aggregated (non-toxic) amyloid beta (circled) can be seen in mice that have been treated with the high dose while in vehicle treated mice, the AR distribution remained thick and linear.

Conclusion:

The reversal of the pathological state in these animals was surprising and unexpected for various reasons. These compounds were designed to bind to misfolded Aβ monomers and prevent them taking on a β-sheet structure, which normally promotes aggregation. Reversal of ongoing Aβ oligomer toxicity indicates that this compound of Formula I, e.g., Compound 1, can additionally reverse toxicity after these oligomers have been formed i.e. this compound is not a classical beta-sheet breaker. Moreover, the extent of the effect was unexpectedly large.

While certain features disclosed herein have been illustrated and described herein, many modifications, substitutions, changes, and equivalents will now occur to those of ordinary skill in the art. It is, therefore, to be understood that the appended claims are intended to cover all such modifications and changes as fall within the true spirit disclosed herein.

1. A method to reverse amyloid β toxicity and rapidly improve function of neuronal, non-neuronal, or neuro-sensory cells, or a combination thereof, in a subject in need, said method comprising administration of a pharmaceutically effective amount of compound of Formula I 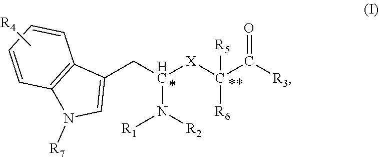
wherein
* refers to a chiral center;
* * refers to a chiral center if R₅ and R₆ are different;
R₁ is hydrogen, —C₁₋₆-alkyl, cycloC₃₋₁₂-alkyl, —C(O)R or —C(O)OR;
R₂ is hydrogen, C₁₋₆-alkyl, or cycloC₃₋₁₂-alkyl;
R₃ is —OR, —NHR, or —N(R)₂,
R₄ is hydrogen, halogen, cyano, trifluoromethyl, —C₁₋₆-alkyl, —C₆₋₁₀-aryl, heteroaryl, —OR, —NHR, —N(R)₂, —C(O)R or —C(O)—NHR;
R⁵ is hydrogen, —C₁₋₆-alkyl or C₂₋₆-alkenyl; or
R⁵ and R⁶ together with the carbon atom carrying them form a cyclic system with 3 to 6 carbon atoms;
R⁶ is hydrogen, —C₁₋₆-alkyl or C₂₋₆-alkenyl;
R⁷ is hydrogen, methyl, ethyl, propyl or cyclopropyl;
R is hydrogen, —C₁₋₆-alkyl, or —C₆₋₁₀-aryl; and
X is a group —C(O)CH₂—, —CH(OH)CH₂—, —CH═CH—, —CH₂—NR—C(O)—, or —C(O)NR;
or an optical isomer, a pharmaceutically acceptable salt, a hydrate, a solvate, or a polymorph thereof.

2. (canceled)

3. The method according to claim 1, wherein the compound of Formula I is selected from Compound 1: 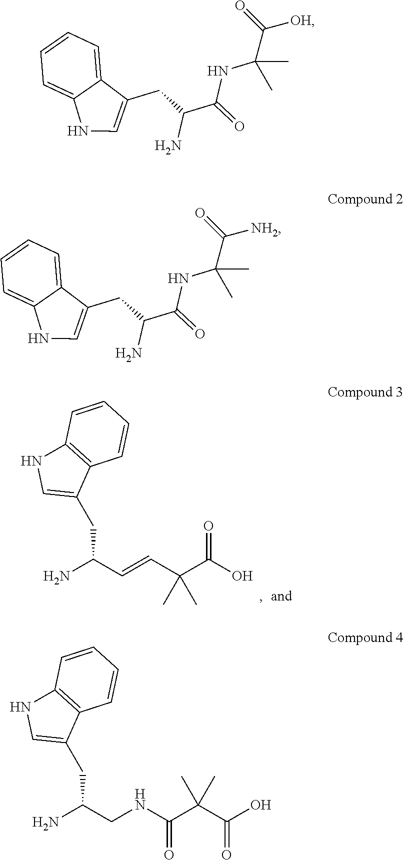
or a pharmaceutically acceptable salt, a hydrate, a solvate, or a polymorph thereof.

4. The method according to claim 1, wherein said rapidly improved function of neuronal, non-neuronal, or neuro-sensory cells, or a combination thereof comprises rapid restoration of impaired neuronal function, or decreased cell death of said neuronal, non-neuronal, or neuro-sensory cells, or a combination thereof.

5. The method according to claim 1, wherein said neuronal, non-neuronal, or neuro-sensory cells comprise retinal ganglion cells (RGC), retinal pigment epithelium (RPE) cells, photosensory cells comprising rod and cone cells, hippocampal cells, or cortical cells, or a combination thereof.

6. The method according to claim 1, wherein said subject is suffering from an amyloid β associated disease and wherein said amyloid-beta associated disease comprises an ophthalmic or a neurological disease or condition.

7. (canceled)

8. The method according to claim 6, wherein said ophthalmic disease or condition comprises primary angle-closure glaucoma, secondary open-angle glaucoma, wide-angle glaucoma, steroid-induced glaucoma, traumatic glaucoma, pigmentary dispersion syndrome, pseudo-exfoliation syndrome, secondary angle-closure glaucoma, neovascular glaucoma, early and intermediate dry (non-exudative) age-related macular degeneration, macular degeneration with geographic atrophy, exudative (“wet”) macular degeneration, or diabetic retinopathy, or a combination thereof and wherein said neurological disease or condition comprises type II diabetes mellitus, diabetes mellitus, Alzheimer's disease (AD), early onset Alzheimer's disease, late onset Alzheimer's disease, presymptomatic Alzheimer's disease, SAA amyloidosis, hereditary Icelandic syndrome, multiple myeloma, medullary carcinoma, aortic medical amyloid, Insulin injection amyloidosis, prion-systemic amyloidosis, chronic inflammation amyloidosis, senile systemic amyloidosis, pituitary gland amyloidosis, hereditary renal amyloidosis, familial British dementia, Finnish hereditary amyloidosis, familial non-neuropathic amyloidosis, and disorders and prion diseases, or a combination thereof.

9. The method according to claim 6, wherein when said amyloid-beta associated disease comprises an ophthalmic disease or condition said rapidly improved cell function comprises one or more aspects of visual function comprising visual acuity, low luminescence vision, contrast sensitivity, cone contrast sensitivity, color vision, focal and general retinal light sensitivity in photopic mesopic (light adaptation) and scotopic (dark adaptation) conditions, and postural stability balance and mobility, in said subject.

10. (canceled)

11. The method according to claim 8, wherein when said neurological disease comprises Alzheimer's disease (AD), early onset Alzheimer's disease, late onset Alzheimer's disease, or pre-symptomatic Alzheimer's disease, said rapid restoration of function comprises improvement of cognitive deficiencies, improvement of memory loss, reduction of abnormal behavior, reduction of hallucinations, reduction of loss of spatial orientation, reduction of apraxia, reduction of aggression, improvement in the ability to perform activities of daily living, or other symptoms of dementia, or any combination thereof, in said subject.

12. The method according to claim 1, wherein said administration comprises oral, topical, nasal, intravenous, subcutaneous, implanted slow-release depots, direct injection using an in-dwelling catheter, intrathecal injection, or intraocular injection administration wherein said administration is in the form of multiple doses administered over a period of time, wherein said time period comprises days, weeks, months, or years, or the lifetime of said subject, and wherein the pattern of dosage within the time period may be at regular intervals, irregular intervals, or a combination thereof comprising administration at regular and irregular intervals.

13. (canceled)

14. (canceled)

15. (canceled)

16. The method according to claim 12, wherein individual doses of said multiple doses each comprise 100% or greater of the therapeutically effective dose, 75-100% of the therapeutically effective dose, or 20-75% of the therapeutically effective dose, or any combination thereof.

17. (canceled)

18. The method according to claim 1, wherein said compound of Formula I is comprised in a pharmaceutically acceptable composition.

19. A method to reverse amyloid β toxicity and rapidly restore the function of neuronal, non-neuronal, or neuro-sensory cells, or a combination thereof, in a subject in need, said method comprising administration of a pharmaceutically effective amount of a non-toxic, non-β-sheet, amorphous amyloid β cluster, said cluster comprises amyloid β₁₋₄₂ and compound of Formula I, wherein the compound of Formula I is represented by the following structure 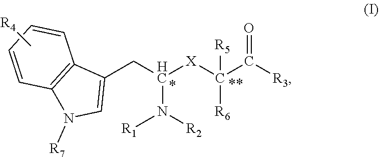
wherein
* refers to a chiral center;
** refers to a chiral center if R₅ and R₆ are different;
R₁ is hydrogen, —C₁₋₆-alkyl, cycloC₃₋₁₂-alkyl, —C(O)R or —C(O)OR;
R₂ is hydrogen, C₁₋₆-alkyl, or cycloC₃₋₁₂-alkyl;
R₃ is —OR, —NHR or —N(R)₂;
R₄ is hydrogen, halogen, cyano, trifluoromethyl, —C₁₋₆-alkyl, —C₆₋₁₀-aryl, heteroaryl, —OR, —NHR, —N(R)₂, —C(O)R or —C(O)—NHR;
R⁵ is hydrogen, —C₁₋₆-alkyl or C₂₋₆-alkenyl; or
R⁵ and R⁶ together with the carbon atom carrying them form a cyclic system with 3 to 6 carbon atoms;
R⁶ is hydrogen, —C₁₋₆-alkyl or C₂₋₆-alkenyl;
R⁷ is hydrogen, methyl, ethyl, propyl or cyclopropyl;
R is hydrogen, —C₁₋₆-alkyl, or —C₆₋₁₀-aryl; and
X is a group —C(O)CH₂—, —CH(OH)CH₂—, —CH═CH—, —CH₂—NR—C(O)—, or —C(O)NR;
or an optical isomer, a pharmaceutically acceptable salt, a hydrate, a solvate, or a polymorph thereof.

20. (canceled)

21. (canceled)

22. The method according to claim 19, wherein the compound of Formula I is selected from Compound 1: 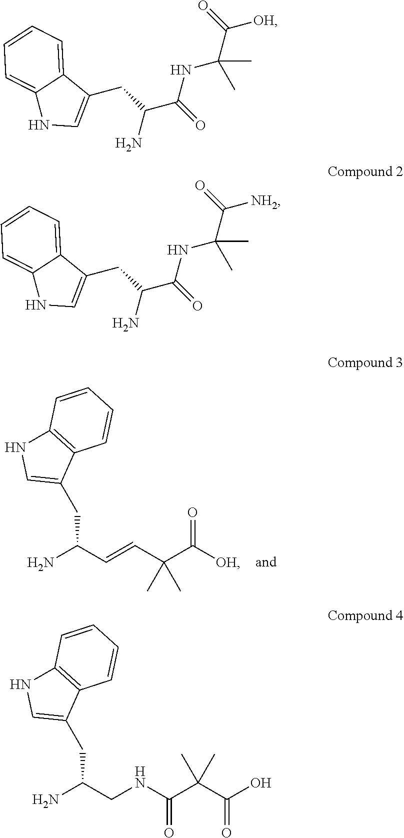
or a pharmaceutically acceptable salt, a hydrate, a solvate, or a polymorph thereof.

23. The method according to claim 19, wherein said reversal of amyloid β toxicity and rapid functional restoration of said neuronal, non-neuronal, or neuro-sensory cells, or a combination thereof results in rapid restoration of impaired neuronal function, or decreased cell death of said neuronal, non-neuronal, or neuro-sensory cells, or a combination thereof.

24. The method according to claim 19, wherein said neuronal, non-neuronal, and neuro-sensory cells comprise retinal ganglion cells (RGC), retinal pigment epithelium (RPE) cells, photosensory cells comprising rod cells and cone cells, hippocampal cells, or cortical cells, or a combination thereof.

25. The method according to claim 19, wherein said subject is suffering from an amyloid β associated disease and wherein said amyloid-beta associated disease comprises an ophthalmic or a neurological disease or condition.

26. (canceled)

27. The method according to claim 25, wherein said ophthalmic disease or condition comprises primary angle-closure glaucoma, secondary open-angle glaucoma, wide-angle glaucoma, steroid-induced glaucoma, traumatic glaucoma, pigmentary dispersion syndrome, pseudo-exfoliation syndrome, secondary angle-closure glaucoma, neovascular glaucoma, early and intermediate dry (non-exudative) age-related macular degeneration, macular degeneration with geographic atrophy, exudative (“wet”) macular degeneration, or diabetic retinopathy, or a combination thereof and wherein said neurological disease or condition comprises type II diabetes mellitus, diabetes mellitus, Alzheimer's disease (AD), early onset Alzheimer's disease, late onset Alzheimer's disease, presymptomatic Alzheimer's disease, SAA amyloidosis, hereditary Icelandic syndrome, multiple myeloma, medullary carcinoma, aortic medical amyloid, Insulin injection amyloidosis, prion-systemic amyloidosis, chronic inflammation amyloidosis, senile systemic amyloidosis, pituitary gland amyloidosis, hereditary renal amyloidosis, familial British dementia, Finnish hereditary amyloidosis, familial non-neuropathic amyloidosis, and disorders and prion diseases, or a combination thereof.

28. The method according to claim 25, wherein when said amyloid-beta associated disease comprises an ophthalmic disease or condition, said rapid restoration of function improves one or more aspects of visual function, said aspects of visual function comprising visual acuity, low luminescence vision, contrast sensitivity, cone contrast sensitivity, color vision, focal and general retinal light sensitivity in photopic mesopic (light adaptation) and scotopic (dark adaptation) conditions, and postural stability balance and mobility, in said subject.

29. (canceled)

30. The method according to claim 27, wherein when said neurological disease comprises Alzheimer's disease (AD), early onset Alzheimer's disease, late onset Alzheimer's disease, or pre-symptomatic Alzheimer's disease, said rapid restoration of function comprises improvement of cognitive deficiencies, improvement memory loss, reduction of abnormal behavior, reduction of hallucinations, reduction of loss of spatial orientation, reduction of apraxia, reduction of aggression, improvement in the ability to perform activities of daily living, or other symptoms of dementia, or any combination thereof, in said subject.

31. The method according to claim 19, wherein said administration comprises oral, topical, nasal, intravenous, subcutaneous, implanted slow-release depots, direct injection using an in-dwelling catheter, intrathecal injection, or intraocular injection administration, wherein said administration is in the form of multiple doses administered over a period of time, wherein said time period comprises days, weeks, months, or years, or the lifetime of said subject and wherein the pattern of dosage within the time period may be at regular intervals, irregular intervals, or a combination thereof comprising administration at regular and irregular intervals.

32. (canceled)

33. (canceled)

34. The method according to claim 19, wherein said non-toxic, non-β sheet amorphous Aβ clusters are comprised in a pharmaceutically acceptable composition.

35. (canceled)

36. (canceled)
